(19) United States
(12) Patent Application Publication
(10) Pub. No.: US 2026/0028643 A1
(43) Pub. Date: Jan. 29, 2026

(54) A CHIMERIC HELPER NUCLEIC ACID MOLECULE COMPRISING HELPER ELEMENTS FROM THREE DISTINCT HELPER VIRUSES

(71) Applicant: POLYPLUS TRANSFECTION, ILLKIRCH-GRAFFENSTADEN (FR)

(72) Inventors: Eric MAURO, LILLE (FR); Sylvain JULIEN, CROIX (FR)

(21) Appl. No.: 18/996,295

(22) PCT Filed: Sep. 29, 2023
(86) PCT No.: PCT/EP2023/077163

§ 371 Date: Jan. 17, 2025

(30) Foreign Application Priority Data
Sep. 30, 2022 (EP) 22306462.7

(51) Int. Cl. C12N 15/86; C07K 14/005

(52) U.S. Cl. CPC C12N 15/86; C07K 14/005; C12N 2710/10322; C12N 2710/10343; C12N 2710/16622; C12N 2750/14152; C12N 2750/14322

(57) ABSTRACT

A chimeric helper nucleic acid molecule contains polynucleotides encoding helper functions from three distinct viruses for producing recombinant adeno-associated virus (rAAV). The molecule includes adenoviral elements with an E2A gene, an E4 gene, and a VA-RNA polynucleotide; a herpes simplex virus (HSV) polynucleotide encoding a UL12 protein or ICP8 protein; and a human bocavirus (HBoV) polynucleotide encoding a NS2 protein or NP1 protein. These polynucleotides are arranged as individual expression cassettes under non-endogenous promoter control. The chimeric helper molecule, when used in combination with an AAV transfer plasmid and Rep/Cap plasmid, provides enhanced production of infectious rAAV particles compared to conventional helper plasmids containing only adenoviral elements. The molecule enables efficient production of multiple AAV serotypes across different cell lines and transfection conditions.

### CROSS REFERENCE TO RELATED APPLICATIONS

This application is the National Phase of PCT International Application No. PCT/EP2023/077163, filed on Sep. 29, 2023, which claims priority under 35 U.S.C. 119(a) to Patent Application No. 22306462.7, filed in Europe on Sep. 30, 2022, all of which are hereby expressly incorporated by reference into the present application.

### REFERENCE TO AN ELECTRONIC SEQUENCE LISTING

The contents of the electronic sequence listing (7141-0038_SequenceListing.xml; Size: 121,131 bytes; and Date of Creation: Jan. 16, 2025) is herein incorporated by reference in its entirety.

The present invention relates to a chimeric helper nucleic acid molecule comprising helper elements from three distinct helper viruses, helper-free virus methods and systems for producing infectious recombinant adeno-associated virus (rAAV). The present invention is directed to a chimeric helper nucleic acid molecule suitable for use in a method of producing infectious rAAV comprising one or more polynucleotides encoding adenoviral helper-viral functions, wherein the polynucleotide(s) comprise(s) the E2A gene from an adenovirus (Ad), the E4 gene from an Ad, and a virus-associated-ribonucleic acid (VA-RNA) polynucleotide from Ad; and one or more polynucleotides comprising an Open Reading frame (ORF) from a gene of a herpes simplex virus (HSV), in particular a polynucleotide encoding at least the UL12 protein from a HSV; and an ORF from a gene of a human bocavirus (HBoV), in particular a polynucleotide encoding at least the NS2 protein from a HBoV, wherein the polynucleotide(s) encode(s) further helper-viral functions and wherein the polynucleotide(s) further comprise(s) non-endogenous sequences for the control of the expression of the ORFs. The present invention also relates to packaging systems, rAAV producer cells, a helper-free virus method of producing a rAAV producing cell line, a method of producing a replication defective infectious rAAV vector particle, and in vitro use of rAAV producer cells in the production of a replication defective infectious rAAV vector particle.

Genetic diseases are caused by absent or defective genes. Gene therapy aims to treat these conditions by delivering a functional copy of the affected gene into a patient's cells. Gene delivery can be performed by recombinant viruses, such as Adeno-associated virus (AAV). AAVs are favoured due to their safety, low pathogenicity and their ability to infect multiple and selective tissues.

Genetic defects are usually present in every cell in a patient's body. Therefore, gene therapy AAVs must be delivered to the majority of cells in an organ to achieve a therapeutic effect. As a result, AAV-based treatments require the administration of very high amounts of virus. Producing sufficient AAV, at an acceptable cost, without compromising safety is a major challenge in bio-production. Increasing or refining AAV production would enable the treatment of a wide variety of genetic diseases. Optimization strategies have focused on the genetic components needed for AAV assembly, the production cell lines, as well as omics-based approaches (Cell Culture Engineering and Technology, 335-364, Chapter Bio-Production of Adeno-Associated Virus for Gene Therapy).

In cultured cells, AAV replication requires co-infection with a helper virus. In the absence of helper virus co-infection AAV can integrate its genome site specifically into the AAVS1 region of chromosome 19. Upon subsequent infection with a helper virus, the AAV genome is released from chromosome 19 by a process termed rescue, and productive replication ensues. The AAV genome cloned into a plasmid vector can also serve to initiate productive AAV replication. When such constructs are transfected into cells and those cells are simultaneously or subsequently infected with a helper virus, the AAV genome is released from the plasmid (J. Virol., 2003, 77 (21), 11480-11490).

AAV, a member of the parvovirus family, contains a single-stranded genome of approximately 4,700 bases. The genome of AAV contains two genes: one coding for non-structural proteins (Rep gene) and the other for the structural proteins (Cap gene). Rep gene encodes for four Rep proteins, namely Rep78, Rep68, Rep50 and Rep42 which are mainly involved in viral DNA replication, encapsidation integration and transcription. Cap gene encodes three proteins which are the building blocks of the mature capsid (VP1, VP2 and VP3), a protein involved in capsid assembly from VP proteins (Assembly Activating Protein) and finally a recent discovered protein involved in viral egress (Membrane Associated Accessory Protein).

The life cycle of AAV is dependent on the presence or absence of a helper virus. The helper virus induces the active replication of the viral genome and the formation of newly infectious AAV, and is, therefore, required for the production of abundant amounts of rAAV. Initially, the helper elements were supplied from an infectious Adenovirus which was used to transduce cells that were transfected with a plasmid encoding the Rep and Cap genes. However, infectious adenoviruses were found in the purified final vector stock of rAAV. To overcome this problem, the minimal helper elements of adenovirus were identified and cloned into a plasmid, giving birth to helper virus-free production methods such as the so-called triple transfection method. Helper plasmid engineering and optimization can have drastic effects on rAAV production efficiency, also depending on AAV serotype, since some helper viruses are found preferentially with specific serotypes, based on sero-epidemiological datas.

U.S. Pat. No. 11,142,775 patent describes a helper virus-free method to produce rAAV or to prepare helper virus-free chimeric RAAV/Human Bocavirus 1 (HBoV1).

The combination of HBoV1 NP1 and NS2 genes with Ad helper genes to create a dual helper plasmid for rAAV vector production in a conventional three-plasmid transfection system has been reported (Mol. Ther. 2018, 11, 40-51).

US20220073947 patent application discloses a circular nucleic acid comprising at least one AAV promoter which is capable of being activated by at least one helper polypeptide or helper polynucleotide originating or derived from a virus selected from Adenoviridae and Herpesviridae.

The international patent application WO2022/173944 discloses a method of producing an AAV in an E1 complementary producer cell comprising the step of transfecting said producer cell with one or more vectors comprising an E1A Ad helper gene; an Ad helper gene selected from E2A, E4, or both; a viral-associated, non-coding RNA (VA RNA); and an AAV gene selected from Rep, Cap, or both.

Examples of commercially available helper plasmids (pHelper) are pALD-X80 by Aldevron, pALD-HELP by Aldevron and VPK-402 by Cellbiolabs.

rAAV are currently the most widely used viral vector for in vivo gene therapy. The production of rAAV is based on cell culture and a defined set of genetic elements including a transgene and structural/non-structural elements used for rAAV production.

Despite years of bioprocessing optimization to improve both quantity and quality of the rAAV production methods, it still remains a challenge to meet the requirements for rAAV-based therapy at sufficient scale and cost. Thus it is an object of the present invention to provide a helper virus-free method for production of rAAV, wherein the method is capable of producing high titers and quality rAAV. It is another object of the present invention to develop a versatile helper nucleic acid molecule, e.g. helper plasmid (pHelper) suitable for production of different AAV serotypes and for different producer cell lines.

In the present invention, the inventors describe the design and the use of a novel helper nucleic acid molecule, and illustrate such helper plasmid for use for the production of rAAV using the triple transfection method in mammalian cells. This new helper nucleic acid molecule, e.g. helper plasmid, contains genes originating from 3 different helper viruses, herein adenovirus, bocavirus and herpesvirus. This novel helper nucleic acid molecule, e.g. helper plasmid, led to an enhanced production of infectious rAAV compared to plasmids containing only adenovirus genes or even adenovirus and bocavirus, or adenovirus and herpes virus genes on the same helper nucleic acid molecule, e.g. helper plasmid. Moreover, this novel nucleic acid molecule, e.g. helper plasmid, proved to be compatible with various AAV serotypes (2, 5, 8 and 9), packaging cell lines (HEK-293T, VPC 2.0 and Expi293F) and also transfection reagents (FectoVIR-AAV, PEIpro and TransIT-VirusGEN), highlighting the high versatility of the designed helper nucleic acid molecule in various production systems.

In particular, the present invention is illustrated with the triple transfection method of mammalian cells, where three distinct plasmids are used: AAV Transfer plasmid (pTransfer) containing a gene of interest flanked by AAV2 inverted terminal repeats (ITR), Rep/Cap plasmid (pRC) containing Rep gene from AAV2 and Cap gene from the desired AAV serotype to be produced, and Helper plasmid (pHelper) containing helper elements from Helper viruse(s) (i.e., Adenovirus, Bocavirus and Herpesvirus) which support AAV production.

In an embodiment, the inventors describe a method using a new type of pHelper that replaces the current pHelpers used for rAAV production. This new pHelper was transfected in the same conditions as the classical methods for the triple transfection but led to better infectious AAV particles titers yields compared to other pHelpers.

The combination of Helper elements from three distinct viruses on the same helper nucleic acid molecule, in particular pHelper (i.e., Adenovirus, Bocavirus and Herpesvirus), enabled the production of highly infectious viral yields in all conditions tested (changing AAV serotype, transfection reagent and packaging cell line).

The present invention relates to a chimeric helper nucleic acid molecule suitable for use in a method of producing infectious recombinant adeno-associated virus (rAAV) comprising:
one or more polynucleotides encoding adenoviral helper-viral (auxiliary) functions for the activation of the expression of Rep and Cap proteins from an adeno-associated virus, wherein the polynucleotide(s) comprise(s) the E2A gene from an adenovirus (Ad), preferably from an adenovirus of serotype 5 (Ad5), the E4 gene, in particular the E4orf6 sequence, from an Ad, preferably from Ad5; and a virus-associated-ribonucleic acid (VA-RNA) polynucleotide from an Ad, preferably from Ad5; and/or
one or more polynucleotides encoding helper-viral functions for AAV production, wherein the polynucleotide(s) comprise(s) an Open Reading frame (ORF) from a gene of a herpes simplex virus (HSV), preferably a herpes simplex virus of serotype 1 (HSV1), and an ORF from a gene of a human bocavirus (HBoV), preferably a human bocavirus of serotype 1 (HBoV1), and wherein the polynucleotide(s) further comprise(s) endogenous or non-endogenous sequences for the control of the expression of the ORFs.

In a particular embodiment, the present invention relates to a chimeric helper nucleic acid molecule suitable for use in a method of producing infectious recombinant adeno-associated virus (rAAV) comprising:
(i) an ORF from a gene of a HSV consisting of a polynucleotide encoding at least one protein of a HSV, in particular a polynucleotide encoding at least the UL12 protein from a HSV or at least the ICP8 protein from a HSV, preferably a polynucleotide encoding the UL12 protein from a HSV or the ICP8 protein from a HSV, preferably from HSV1;
(ii) an ORF from a gene of a HBoV consisting of a polynucleotide encoding the NS2 protein from a HBoV or the NP1 protein from a HBoV, preferably from HBoV1;
(iii) the VA-RNA polynucleotide from an Ad, preferably from Ad5;
(iv) the E2A gene from an Ad, preferably from Ad5;
(v) the E4 gene, in particular the E4orf6 sequence, from an Ad, preferably from Ad5;
wherein each of the (i) (ii), (iii), (iv) and (v) polynucleotides are inserted as individual expression cassettes within the chimeric helper nucleic acid molecule, and wherein polynucleotides of (i) to (v) are arranged in any order and/or orientation with respect to one another;
with the proviso that the chimeric helper nucleic acid molecule does not comprise (ii) an ORF from a gene of a HBoV consisting of a polynucleotide encoding the NS2 protein from a HBoV and the NP1 protein from a HBoV.

In a particular embodiment of the invention, the chimeric helper nucleic acid molecule suitable for use in a method of producing infectious recombinant adeno-associated virus (rAAV) comprises:
(i) an ORF from a gene of a HSV consisting of a polynucleotide encoding at least one protein of a HSV, preferably one or two proteins of a HSV, in particular a polynucleotide encoding at least the UL12 protein from a HSV or the at least ICP8 protein from a HSV, preferably a polynucleotide encoding the UL12 protein from a HSV and/or the ICP8 protein from a HSV, preferably from HSV1;
(ii) an ORF from a gene of a HBoV consisting of a polynucleotide encoding the NS2 protein from a HBoV or the NP1 protein from a HBoV, preferably from HBoV1;
(iii) the VA-RNA polynucleotide from an Ad, preferably from Ad5;
(iv) the E2A gene from an Ad, preferably from Ad5;
(v) the E4 gene, in particular the E4orf6 sequence, from an Ad, preferably from Ad5;
wherein each of the (i) (ii), (iii), (iv) and (v) polynucleotides are inserted as individual expression cassettes within the chimeric helper nucleic acid molecule, and wherein polynucleotides of (i) to (v) are arranged in any order and/or orientation with respect to one another;
with the proviso that the chimeric helper nucleic acid molecule does not comprise (ii) an ORF from a gene of a HBoV consisting of a polynucleotide encoding the NS2 protein from a HBoV and the NP1 protein from a HBoV.

As defined herein, the term “chimeric nucleic acid molecule” refers to a nucleic acid molecule comprising sequences of different organisms.

As defined herein, the term “nucleic acid molecule” or “nucleic acid” refers to a polymeric compound of covalently linked nucleotides thereby forming a strand-based structure. A nucleic acid is either single-, or double-stranded, or combined single and double-stranded on distinct regions of the nucleic acid strand; and is selected from the group consisting of deoxyribonucleic acid (DNA), ribonucleic acid (RNA), small interfering RNA (siRNA), asymmetrical interfering RNA (aiRNA), dicer-substrate short interfering RNA (dsiRNA), small hairpin RNA (shRNA), RNA transcripts, microRNA (miRNA), messenger RNA (mRNA), circular RNA (circRNA), guide RNA (gRNA), small activating RNA (saRNA), small regulatory RNA (srRNA), long non-coding (lncRNA) and antisense oligonucleotide. The term “polynucleotide” may be used interchangeably with the term “nucleic acid molecule”. Particular nucleic acid molecules are Open Reading Frames (ORF) that encode the amino acid sequence of polypeptides and may express such polypeptides when placed under the control of regulatory elements for transcription and translation, including a promoter.

As defined herein, the term “helper” or “helper function” or “helper functions” when referring to a helper nucleic acid molecule suitable for AAV production means that the molecule provides function(s), i.e., “helper-viral functions” that are required for AAV to be replicated and for viral gene expression for transduction and for viral particles assembly, i.e., for AAV genome to be packaged in a cell, in particular a mammalian cell (Marie-Claude Geoffroy, et al. Current Gene Therapy, 2005, 5(3), 265-271). In an embodiment, such helper function(s) may be brought by a polypeptide encoded by the nucleic acid molecule. In an embodiment, such helper function(s) when brought by the nucleic acid molecule originating from HSV or HBoV may be of the same nature as a helper function brought by the Ad nucleic acid used according to the invention, in particular by the E2A, E4 and/or VA-RNA. In an embodiment the helper function(s) when brought by the nucleic acid molecule originating from HSV or HBoV may enhance or improve the helper functions brought by the Ad nucleic acid used according to the invention i.e., may enable increasing the rAAV virus titer in the producer cells and/or the transducing titer of the produced rAAV (TU/ml or VG/ml) determined after infection of permissive cells. In an embodiment, the helper function(s) when brought by the nucleic acid molecule originating from HSV or HBoV may be functions that provide new activities in the producer cells and/or the infected cells with respect to those brought by the Ad nucleic acid used according to the invention. Such new activities may also lead to an increase in the rAAV titer in the producer cells and/or the transducing titer of the produced rAAV (TU/ml or VG/ml) determined after infection of permissive cells (such as HT-1080 or CHO-K1). The increase in the rAAV titer may be assessed or measured by any appropriate known methods by comparison to the titer of the produced rAAV obtained when the nucleic acid molecule originating from HSV and HBoV are not used according to the invention. Examples of the measurement of the rAAV titer are disclosed in the Examples herein. In an embodiment, the helper function(s) provided by the HSV or HBoV helper genes or ORF improve the versatility of the produced rAAV with respect to the producer cells, transfection agent and/or rAAV serotype (in particular considering serotypes 2, 5, 8 and/or 9). The rAAV titer may be determined as disclosed in the examples using the Capsid titer (VP/ml), e.g., using a titration ELISA kit, the genome titer (VG/ml) by qPCR performed on a reporter gene product.

As defined herein, the term “expression cassette” refers to a functional expression unit capable of driving the expression of one or more incorporated polynucleotides.

As defined herein, the term “VA-RNA from an Ad” refers to the VA-RNA element from an Ad, which is a small non-coding RNA transcribed by pol III. In a preferred embodiment, it refers to VA-RNA from Ad5 (from position 10,545 to position 11,029 of the NCBI reference sequence AC_000008.1).

As defined herein, the term “E2A gene from an Ad” refers to the E2A gene that encodes for the E2A protein from an Ad transcribed by pol III. In a preferred embodiment, it refers to E2A gene from Ad5 (from position 22,388 to position 27,121 of the NCBI reference sequence AC_000008.1).

As defined herein, the term “E4orf6 sequence from an Ad” refers to E4orf6, which is one of the proteins encoded by the E4 gene from an Ad. In a preferred embodiment, it refers to the E4orf6 coding sequence from Ad5 (from position 33, 188 to position 34,072 of the NCBI reference sequence AC_000008.1).

The inventors have shown that the use of the triple chimeric helper elements according to the invention, advantageously provide a higher number of infectious recombinant viruses of various AAV serotypes (in particular of the serotypes 2, 5, 8 and/or 9) with different producer cells thereby showing versatility. The inventors have also shown that the infectious titer of the produced rAAV (measured as TU/VP/VG) may significantly be increased using the triple chimeric helper elements of the invention. Accordingly, the helper elements brought by the nucleic acid molecule originating from HSV and HBoV are regarded as providing further helper-viral functions with respect to the helper-viral functions brought by the Ad nucleic acid molecules.

The expression “originating” or “originates” in plural or singular used in the present description in particular to disclose the nucleic acid molecules originating from HSV or HBoV viruses refers to the fact that the expressed polypeptide encoded by the nucleic acid originating from HSV or HBoV viruses are characteristic of a native viral proteins expressed by these respective viruses to the extent that they confer similar or identical effect as helper elements when used in the present invention. The nucleotide sequence of such nucleic acid molecules may be identical to the wild-type sequence found in the genome of the virus or may be modified with respect to such wild-type sequence especially modified for optimization of expression in a determined producer cell. The nucleic acid molecules originating from HSV and HBoV viruses may be a purified or a synthetic molecule.

As defined herein, the term “recombinant adeno-associated virus (rAAV)” refers to an AAV virus comprising in its genome one or more heterologous genes or polynucleotides, i.e., which are not of AAV origin. A rAAV may accordingly be used as a vector to enable expression of the heterologous gene or polynucleotide in target cells.

As defined herein, the term “gene” refers to a nucleic acid molecule or Open reading Frame (ORF) which is transcribed (in the case of DNA) and translated (involving production of a mRNA) into a polypeptide in vitro or in vivo under the control of its endogenous or native regulatory sequences such as promoters.

In a particular embodiment of the invention, the chimeric helper nucleic acid molecule is a recombinant helper nucleic acid molecule.

As defined herein, the term “recombinant nucleic acid molecule” means that the nucleic acid molecule has been genetically altered, e.g., by the addition or insertion of a heterologous nucleic acid construct into the molecule.

In another particular embodiment of the invention, the chimeric helper nucleic acid molecule is a chimeric helper expression vector, in particular a chimeric helper expression plasmid.

As defined herein, the term “vector” relates to biological or chemical entities suitable for the delivery of the polynucleotides of interest, in particular polynucleotides encoding polypeptides of interest to cells, in particular to producer cells as defined herein, or to target cells. The term “vector” hence refers to, or comprises in its structure, a nucleic acid molecule comprising genetic information and capable of transmitting nucleic acid molecules bearing said genetic information. In some embodiments, a vector enables expressing said nucleic acid molecules bearing genetic information, in particular enables expression of polypeptides from said molecules. In an embodiment, a vector is an expression vector that comprises regulatory elements for the expression of nucleic acid molecules that it comprises, especially to express heterologous polypeptides. In the context of the invention the term “vector” hence relates to the nucleic acid molecules that bear the viral helper functions as disclosed herein or relates to the nucleic acid molecules that bear the transgene of interest such as in the plasmid transfer disclosed herein or relates to the nucleic acid molecules that bear the molecules encoding the Rep and/or Cap proteins such as pRep/Cap and it relates also to the produced recombinant AAV particles. A vector may for example be a replicon or may comprise a replicon. A vector can be episomal or non episomal. In an embodiment, the vector may be a plasmid modified by genetic engineering and intended to transfer nucleic acid molecules into a cell. In an embodiment, a vector may be a recombinant viral particle, the genome of which comprises nucleic acid with genetic information to be transferred into a target cell and when required expressed into such cell. In an embodiment, the vector according to the invention comprises at least a bacterial functional unit (ori) and can also comprise one or more expression units and/or one or more integration units.

As defined herein, the term “plasmid” refers to a DNA molecule, different from chromosomal DNA, capable of autonomous replication. Plasmids are generally circular and double-stranded DNA.

In a particular embodiment of the invention, the rAAV is selected from the group consisting of rAAV of serotype 1 (rAAV1), rAAV of serotype 2 (rAAV2), rAAV of serotype 3 (rAAV3), rAAV of serotype 4 (rAAV4), rAAV of serotype 5 (rAAV5), rAAV of serotype 6 (rAAV6), rAAV of serotype 7 (rAAV7), rAAV of serotype 8 (rAAV8), rAAV of serotype 9 (rAAV9), rAAV of serotype 10 (rAAV10), rAAV of serotype 11 (rAAV11), rAAV of serotype 12 (rAAV12) and rAAV of serotype 13 (rAAV13). In a particular embodiment, the produced rAAV is from a serotype selected among rAAV2, rAAV5, rAAV8 and rAAV9, preferably rAAV2, rAAV5 and rAAV9.

In a particular embodiment of the invention, the Ad providing the polynucleotides encoding polypeptides harbouring helper functions for AAV is an Ad of serotype 2 (Ad2) or an Ad of serotype 5 (Ad5), preferably Ad5. In a particular embodiment, the Ad proves the helper function for polynucleotide encoding the Cap protein of AAV of serotype 2 (AAV2), of serotype 5 (AAV5) or of serotype 9 (AAV9), in particular AAV2.

Herpex Simplex Virus (HSV) is a human herpesvirus that is an encapsidated double-stranded DNA virus classified in two known serotypes HSV-1 (Human alphaherpesvirus 1, also designated as HHV1, of which the complete genome sequence is available at GenBank under the accession number MN136524 together with the amino acid sequences of the annotated encoded proteins) and HSV-2 (Human alphaherpesvirus 2 of which the complete genome sequence is available at EMBL under accession number no. Z86099 together with the amino acid sequences of the annotated encoded proteins). The DNA genome consists of two covalently linked components designated as L (long) and S (short) Unique regions. The HSV virions is a particle comprising a capsid and an envelope. Among the non-structural proteins, HSV encode the UL12 which is an alkaline exonuclease (DNAse) involved in viral nucleic acid metabolism by processing linear or branched viral DNA intermediates to promote the production of mature progeny viral DNA molecules. The protein has endonuclease and exonuclease activities on both double-stranded and single-stranded DNA substrate.

In a particular embodiment of the invention, the ORF from a gene of a HSV consisting of a polynucleotide encoding at least one protein of a HSV is a polynucleotide encoding structural and/or non-structural proteins of HSV, in particular proteins selected from the group consisting of UL5, UL8, UL12, UL29, UL52 and ICP8 of HSV. Preferably the polynucleotide encodes the sole UL12 protein from a HSV, or the UL12 protein from a HSV in a combination with other(s) protein(s) of HSV such as the above-mentioned proteins from a HSV; or the polynucleotide encodes the sole ICP8 protein of a HSV, or the ICP8 protein of a HSV in a combination with other(s) protein(s) of HSV such as the above-mentioned proteins from a HSV. In particular the polynucleotide encodes the UL12 protein from a HSV and the ICP8 protein from a HSV.

In a particular embodiment of the invention, the HSV is a HSV of serotype 1 (HSV1) or a HSV of serotype 2 (HSV2), preferably HSV1. In an embodiment, the polynucleotide encodes the UL12 protein from HSV1. In another embodiment, the polynucleotide encodes the ICP8 protein from HSV1. In another embodiment, the polynucleotide encodes the UL12 and ICP8 proteins from HSV1.

In a preferred embodiment of the invention, the ORF from a gene of a HSV consists of a polynucleotide encoding the UL12 protein from a HSV, preferably from HSV1.

Human bocavirus (HBoV) is a single-stranded DNA virus of the Parvoviridae family encompassing HBoV1, HBoV2, HBoV3 and HBoV4 serotypes, among which some are capable of infecting human being. Several sequences of the virus genome from strains isolated from patients are available in GenBank under accession numbers MG953829, MG953830, MG953831, MG953832, MG953833 and MG953834 together with the amino acid sequences of the encoded proteins. The genome of HBoV encodes 3 ORFs (ORF1, ORF2, ORF3). One of them encodes 4 non-structural proteins (NS1, NS2, NS3 and NS4), wherein one encodes the small non-structural protein NP1 protein and the last one in the genome encodes the capsid proteins (VP1, VP2 and VP3). The NP1 gene is an alternate reading frame to VP1 and overlaps the start of VP1. NP1 has been characterized for its capability to induce apoptosis in cells.

In an embodiment, the helper function(s) provided by the nucleic acid molecule(s) originating from HSV or HBoV are achieved by the expression of polypeptides of respectively HSV or HBoV.

The term “polypeptide” or “protein” used interchangeably defined herein a wild-type or native polypeptide of a virus among the HSV or HBoV viruses or as a fragment of such wild-type polypeptide or as a mutated polypeptide or as a synthetic polypeptide, wherein such fragment, mutant or synthetic polypeptide maintains the helper effect of the wild-type polypeptide (or of the collectively used wild-type polypeptides) on the production of the rAAV in particular on the titer of the produced rAAV. In an embodiment, a fragment or a mutant polypeptide may have more than 80% or more than 90%, in particular more than 95% identity with the wild-type sequence of the polypeptide of reference.

In a particular embodiment of the invention, the polynucleotide(s) encoding further helper-viral functions provided by polynucleotide(s) originating from a HBoV are polynucleotide(s) encoding structural and/or non-structural proteins of HBoV, in particular proteins selected from the group consisting of NS1, NS2, NS3, NS4, NP1, VP1, VP2 and VP3 proteins of HBoV. Preferably, the polynucleotides encode the sole HBoV NS2 protein, or the HBoV NS2 protein in a combination with other(s) protein(s) of HBoV, or encode the sole HBoV NP1 protein, or the HBoV NP1 protein in a combination with other(s) protein(s) of HBoV.

In a preferred embodiment of the invention, the ORF from a gene of a HBoV consists of a polynucleotide encoding the NS2 protein from a HBoV or the NP1 protein from a HBoV. Preferably, the ORF from a gene of a HBoV consists of a polynucleotide encoding the NS2 protein from a HBoV.

In a particular embodiment of the invention, the HBoV is a HBoV of serotype 1 (HBoV1), a HBoV of serotype 2 (HBoV2), a HBoV of serotype 3 (HBoV3) or a HBoV of serotype 4 (HBoV4), preferably HBoV1. In an embodiment, the polynucleotides encode HBoV1 helper proteins, in particular NS2 protein, especially the sole NS2 protein, or a combination of NS2 with other(s) protein(s) such as NP1 of HBoV1. Preferably, the ORF from a gene of a HBoV consists of a polynucleotide encoding the NS2 protein of HBoV1.

In an embodiment, the polynucleotide(s) encoding further helper functions encode the UL12 protein of a HSV serotype and NS2 protein, especially the sole NS2 protein, or a combination of NS2 with other protein such as NP1 of HBoV. In a particular embodiment, the polynucleotides encode the UL12 protein of a HSV1 serotype and NS2 protein, especially the sole NS2 protein, or a combination of NS2 with other(s) protein(s) such as NP1 of HBoV1.

In a particular embodiment of the invention, the ORF from a gene of a HSV consists of a polynucleotide encoding the UL12 protein from a HSV, preferably from HSV1; and the ORF from a gene of a HBoV consists of a polynucleotide encoding the NP1 protein from a HBoV, preferably from HBoV1.

In a preferred embodiment of the invention, the ORF from a gene of a HSV consists of a polynucleotide encoding the UL12 protein from a HSV, preferably from HSV1; and the ORF from a gene of a HBoV consists of a polynucleotide encoding the NS2 protein from a HBoV, preferably from HBoV1.

In a particular embodiment of the invention, the ORF from a gene of a HSV consists of a polynucleotide encoding the ICP8 protein from a HSV, preferably from HSV1; and the ORF from a gene of a HBoV consists of a polynucleotide encoding the NP1 protein from a HBoV, preferably from HBoV1.

In a preferred embodiment of the invention, the ORF from a gene of a HSV consists of a polynucleotide encoding the ICP8 protein from a HSV, preferably from HSV1; and the ORF from a gene of a HBoV consists of a polynucleotide encoding the NS2 protein from a HBoV, preferably from HBoV1.

In a particular embodiment of the invention, the ORF from a gene of a HSV consists of a polynucleotide encoding the UL12 protein from a HSV in a combination with other(s) protein(s) of HSV such as the above-mentioned proteins from a HSV; and the ORF from a gene of a HBoV consists of a polynucleotide encoding the NP1 protein from a HBoV, preferably from HBoV1. More particularly, the ORF from a gene of a HSV consists of a polynucleotide encoding the UL12 protein from a HSV and the ICP8 protein from a HSV, preferably from HSV1; and the ORF from a gene of a HBoV consists of a polynucleotide encoding the NP1 protein from a HBoV, preferably from HBoV1.

In a preferred embodiment of the invention, the ORF from a gene of a HSV consists of a polynucleotide encoding the UL12 protein from a HSV in a combination with other(s) protein(s) of HSV such as the above-mentioned proteins from a HSV; and the ORF from a gene of a HBoV consists of a polynucleotide encoding the NS2 protein from a HBoV, preferably from HBoV1. In particular, the ORF from a gene of a HSV consists of a polynucleotide encoding the UL12 protein from a HSV and the ICP8 protein from a HSV, preferably from HSV1; and the ORF from a gene of a HBoV consists of a polynucleotide encoding the NS2 protein from a HBoV, preferably from HBoV1.

In a particular embodiment of the invention, the nucleic acids (i) and (ii) are under the control of a non-endogenous promoter, in particular a different promoter. In a preferred embodiment of the invention, the NS2 protein from a HBoV, preferably from HBoV1 is under the control of the pCMV promoter. In another preferred embodiment of the invention, the NP1 protein from a HBoV, preferably from HBoV1 is under the control of the pCMV promoter.

In another preferred embodiment of the invention, the at least UL12 protein from a HSV, preferably from HSV1 is under the control of the EFS promoter (i.e., core promoter of the eukaryotic translation elongation factor 1α).

In another preferred embodiment of the invention, the at least ICP8 protein from a HSV, preferably from HSV1 is under the control of the EFS promoter (i.e., core promoter of the eukaryotic translation elongation factor 1α).

In another preferred embodiment of the invention, the at least ICP8 protein from a HSV, in particular from HSV1 and the at least UL12 protein from a HSV, in particular from HSV1, preferably the ICP8 and UL12 proteins from a HSV, in particular from HSV1, are under the control of the EFS promoter (i.e., core promoter of the eukaryotic translation elongation factor 1α).

As defined herein, the term “promoter” refers to a nucleic acid sequence that drives gene expression. Examples of promoters include, but are not limited to, a cytomegalovirus (CMV) promoter, a CMV enhancer, an EFS promoter, a SV40 virus promoter, a CBA promoter, a CAG promoter, an E2A promoter and an E4 promoter.

The term “non-endogenous promoter” refers to a promoter that is from a source different from the native promoter of the encoded sequence.

In a preferred embodiment of the invention, the polynucleotides in the chimeric nucleic acid molecule have the following order from the 5′ to 3′ direction: (i), (ii), (iii), (iv) and (v), and wherein the nucleic acids (i) and (ii) are under the control of a non-endogenous promoter, in particular a different promoter.

In a particular embodiment of the invention, the chimeric helper nucleic acid molecule further comprises additional adenovirus genes, for example the E1 gene from an Ad, preferably from Ad5.

In a particular embodiment of the invention, the chimeric helper nucleic acid molecule further comprises:
an origin of replication (ori); and/or
a herpes simplex virus thymidine kinase (HSV TK polyA) signal downstream from the HSV UL12 ORF; and/or
a bovine growth hormone polyadenylation (bGH polyA) signal downstream from the HBoV NS2 ORF; and/or
a transcription regulation element such as a cytomegalovirus (CMV) promoter, a CMV enhancer, an EFS promoter, a SV40 virus promoter, a CBA promoter, a CAG promoter, an E2A promoter and/or an E4 promoter to control the transcription of the HSV and HBoV ORFs; and/or
optionally a nucleic acid encoding a marker gene such as the kanamycin resistance gene.

As defined herein, the term “origin of replication (ori)” refers to an insulated origin of replication, based on high copy pmB1, and flanked with 2 bacterial transcription terminators, that are T7-terminator in 5′ position and LuxIA terminator in 3′ position. Such transcriptional insulation has been devised to minimize transcriptional interference from neighboring content, hence ensuring higher amplification rates in bacteria.

As defined herein, the term “a nucleic acid encoding the kanamycin resistance gene” refers to an antibiotic resistance cassette, consisting in nptII ORF conferring resistance to kanamycin treatment under the control of pAmpR promoter and terminater by an hairpin terminator.

In a preferred embodiment of the invention, in the chimeric helper nucleic acid molecule:
the polynucleotide encoding the UL12 protein from HSV1 is of SEQ ID NO:1;
the polynucleotide encoding the NS2 protein from HBoV1 is of SEQ ID NO:2;
the VA-RNA polynucleotide from Ad5 is of SEQ ID NO:3;
the E2A gene from Ad5 is of SEQ ID NO:4;
the E4 gene from Ad5 is of SEQ ID NO:5, in particular the E4orf6 sequence from Ad5 is of SEQ ID NO:6; and/or
the polynucleotide encoding the ICP8 protein from HSV1 is of SEQ ID NO:32.

In a preferred embodiment of the invention, in the chimeric helper nucleic acid molecule:
the ori is a nucleotide sequence of SEQ ID NO:7;
the HSV TK polyA signal is a polynucleotide of sequence SEQ ID NO:8;
the bGH polyA signal is a polynucleotide of sequence SEQ ID NO:9;
the CMV promoter is a polynucleotide of sequence SEQ ID NO:10;
the CMV enhancer is a polynucleotide of sequence SEQ ID NO:11;
the EFS promoter is a polynucleotide of sequence SEQ ID NO:12;
the E2A promoter is a polynucleotide of sequence SEQ ID NO:13;
the E4 promoter is a polynucleotide of sequence SEQ ID NO:14; and/or
the kanamycin resistance gene is of sequence SEQ ID NO:15.

In a preferred embodiment of the invention, the chimeric helper nucleic acid molecule comprises (i) a polynucleotide insert comprising the EFS promoter, the polynucleotide encoding the UL12 protein from HSV1 and the HSV TK polyA signal, whose sequence is the sequence of SEQ ID NO:16, and (ii) a polynucleotide insert comprising the CMV promoter, the polynucleotide encoding the NS2 protein from HBoV1 and the bGH polyA signal, whose sequence is the sequence of SEQ ID NO:17.

In a particular embodiment of the invention, the chimeric helper nucleic acid molecule comprises (i) a polynucleotide insert comprising the EFS promoter, the polynucleotide encoding the UL12 protein from HSV1 and the HSV TK polyA signal, whose sequence is the sequence of SEQ ID NO:16, and (ii) a polynucleotide insert comprising the CMV promoter, the polynucleotide encoding the NP1 protein from HBoV1 and the bGH polyA signal, whose sequence is the sequence of SEQ ID NO:17.

In another particular embodiment of the invention, the chimeric helper nucleic acid molecule comprises (i) a polynucleotide insert comprising the EFS promoter, the polynucleotide encoding the ICP8 protein from HSV1 and the HSV TK polyA signal, whose sequence is the sequence of SEQ ID NO:16, and (ii) a polynucleotide insert comprising the CMV promoter, the polynucleotide encoding the NS2 protein from HBoV1 and the bGH polyA signal, whose sequence is the sequence of SEQ ID NO:17.

In another particular embodiment of the invention, the chimeric helper nucleic acid molecule comprises (i) a polynucleotide insert comprising the EFS promoter, the polynucleotide encoding the ICP8 protein from HSV1 and the HSV TK polyA signal, whose sequence is the sequence of SEQ ID NO:16, and (ii) a polynucleotide insert comprising the CMV promoter, the polynucleotide encoding the NP1 protein from HBoV1 and the bGH polyA signal, whose sequence is the sequence of SEQ ID NO:17.

In another particular embodiment of the invention, the chimeric helper nucleic acid molecule comprises (i) a polynucleotide insert comprising the EFS promoter, the polynucleotide encoding the UL12 protein from HSV1 and the HSV TK polyA signal, whose sequence is the sequence of SEQ ID NO:16, (ii) a polynucleotide insert comprising the EFS promoter, the polynucleotide encoding the ICP8 protein from HSV1 and the HSV TK polyA signal, whose sequence is the sequence of SEQ ID NO:16, and (iii) a polynucleotide insert comprising the CMV promoter, the polynucleotide encoding the NS2 protein from HBoV1 and the bGH polyA signal, whose sequence is the sequence of SEQ ID NO:17.

In another particular embodiment of the invention, the chimeric helper nucleic acid molecule comprises (i) a polynucleotide insert comprising the EFS promoter, the polynucleotide encoding the UL12 protein from HSV1 and the HSV TK polyA signal, whose sequence is the sequence of SEQ ID NO:16, (ii) a polynucleotide insert comprising the EFS promoter, the polynucleotide encoding the ICP8 protein from HSV1 and the HSV TK polyA signal, whose sequence is the sequence of SEQ ID NO:16, and (iii) a polynucleotide insert comprising the CMV promoter, the polynucleotide encoding the NP1 protein from HBoV1 and the bGH polyA signal, whose sequence is the sequence of SEQ ID NO:17.

In a preferred embodiment of the invention, the chimeric helper nucleic acid molecule is a plasmid, in particular the plasmid Ad-HBoV-HSV of SEQ ID NO:18, or the plasmid Ad-HBoV-HSV5 of SEQ ID NO:35, preferably is the plasmid Ad-HBoV-HSV of SEQ ID NO:18.

In a preferred embodiment of the invention, in the chimeric helper nucleic acid molecule:
the amino acid sequence of the UL12 protein from HSV1 is of SEQ ID NO:19;
the amino acid sequence of the NS2 protein from HBoV1 is of SEQ ID NO:20;
the amino acid sequence of the E2A protein from Ad5 is of SEQ ID NO:21;
the amino acid sequence of the E4orf1 sequence from Ad5 is of SEQ ID NO:22;
the amino acid sequence of the E4orf6 sequence from Ad5 is of SEQ ID NO:23;
the nucleotide sequence encoding the NP1 protein from HBoV1 is of SEQ ID NO:24;
the amino acid sequence of the NP1 protein from HBoV1 is of SEQ ID NO:25;
the E1 gene from Ad5 is a polynucleotide of SEQ ID NO:26;
the amino acid sequence of the E1A protein from Ad5 is of SEQ ID NO:27;
the amino acid sequence of the E1B55k protein from Ad5 is of SEQ ID NO:28;
the amino acid sequence of the E4orf2 sequence from Ad5 is of SEQ ID NO:29;
the amino acid sequence of the E4orf3 sequence from Ad5 is of SEQ ID NO:30;
the amino acid sequence of the E4orf4 sequence from Ad5 is of SEQ ID NO:31;
the amino acid sequence of the E4 protein from Ad5 is of SEQ ID NO:22, SEQ ID NO:29, SEQ ID NO:30 or SEQ ID NO:31;
the amino acid sequence of the E1 protein from Ad5 is of SEQ ID NO:27 or SEQ ID NO:28; and/or
the amino acid sequence of the ICP8 protein from HSV1 is of SEQ ID NO:33.

The E4 protein may consist of the E4orf1 sequence, the E4orf2 sequence, the E4orf3 sequence, the E4orf4 sequence and the E4orf6 sequence as defined above.

The E1 protein may consist of the E1A protein and the E1B55k protein as defined above.

In a preferred embodiment of the invention, the chimeric helper nucleic acid molecule comprises:
(i) the ORF from a gene of a HSV consisting of a polynucleotide encoding the UL12 protein from a HSV, preferably from HSV1;
(ii) the ORF from a gene of a HBoV consisting of a polynucleotide encoding the NS2 protein from a HBoV, preferably from HBoV1;
(iii) the VA-RNA polynucleotide from an Ad, preferably from Ad5;
(iv) the E2A gene from an Ad, preferably from Ad5;
(v) the E4 gene, in particular the E4orf6 sequence, from an Ad, preferably from Ad5;
wherein each of the (i) (ii), (iii), (iv) and (v) polynucleotides are inserted as individual expression cassettes within the chimeric helper nucleic acid molecule, and wherein polynucleotides of (i) to (v) are arranged in any order and/or orientation with respect to one another; preferably wherein each of the (i) (ii), (iii), (iv) and (v) polynucleotides are inserted as individual expression cassettes within the chimeric helper nucleic acid molecule, wherein the polynucleotides in the chimeric nucleic acid molecule have the following order from the 5′ to 3′ direction: (i), (ii), (iii), (iv) and (v), and wherein the nucleic acids (i) and (ii) are under the control of a non-endogenous promoter, in particular a different promoter;
with the proviso that the chimeric helper nucleic acid molecule does not comprise (ii) an ORF from a gene of a HBoV consisting of a polynucleotide encoding the NS2 protein from a HBoV and the NP1 protein from a HBoV; and
with the proviso that the chimeric helper nucleic acid molecule does not comprise further polynucleotides encoding proteins from a HSV.

In another preferred embodiment of the invention, the chimeric helper nucleic acid molecule comprises:
(i) the ORF from a gene of a HSV consisting of a polynucleotide encoding the UL12 protein from a HSV, preferably from HSV1;
(ii) the ORF from a gene of a HBoV consisting of a polynucleotide encoding the NP1 protein from a HBoV, preferably from HBoV1;
(iii) the VA-RNA polynucleotide from an Ad, preferably from Ad5;
(iv) the E2A gene from an Ad, preferably from Ad5;
(v) the E4 gene, in particular the E4orf6 sequence, from an Ad, preferably from Ad5;
wherein each of the (i) (ii), (iii), (iv) and (v) polynucleotides are inserted as individual expression cassettes within the chimeric helper nucleic acid molecule, and wherein polynucleotides of (i) to (v) are arranged in any order and/or orientation with respect to one another; preferably wherein each of the (i) (ii), (iii), (iv) and (v) polynucleotides are inserted as individual expression cassettes within the chimeric helper nucleic acid molecule, wherein the polynucleotides in the chimeric nucleic acid molecule have the following order from the 5′ to 3′ direction: (i), (ii), (iii), (iv) and (v), and wherein the nucleic acids (i) and (ii) are under the control of a non-endogenous promoter, in particular a different promoter;
with the proviso that the chimeric helper nucleic acid molecule does not comprise (ii) an ORF from a gene of a HBoV consisting of a polynucleotide encoding the NS2 protein from a HBoV and the NP1 protein from a HBoV, and
with the proviso that the chimeric helper nucleic acid molecule does not comprise further polynucleotides encoding proteins from a HSV.

In another preferred embodiment of the invention, the chimeric helper nucleic acid molecule comprises:
(i) the ORF from a gene of a HSV consisting of a polynucleotide encoding the ICP8 protein from a HSV, preferably from HSV1;
(ii) the ORF from a gene of a HBoV consisting of a polynucleotide encoding the NS2 protein from a HBoV, preferably from HBoV1;
(iii) the VA-RNA polynucleotide from an Ad, preferably from Ad5;
(iv) the E2A gene from an Ad, preferably from Ad5;
(v) the E4 gene, in particular the E4orf6 sequence, from an Ad, preferably from Ad5;
wherein each of the (i) (ii), (iii), (iv) and (v) polynucleotides are inserted as individual expression cassettes within the chimeric helper nucleic acid molecule, and wherein polynucleotides of (i) to (v) are arranged in any order and/or orientation with respect to one another; preferably wherein each of the (i) (ii), (iii), (iv) and (v) polynucleotides are inserted as individual expression cassettes within the chimeric helper nucleic acid molecule, wherein the polynucleotides in the chimeric nucleic acid molecule have the following order from the 5′ to 3′ direction: (i), (ii), (iii), (iv) and (v), and wherein the nucleic acids (i) and (ii) are under the control of a non-endogenous promoter, in particular a different promoter;
with the proviso that the chimeric helper nucleic acid molecule does not comprise (ii) an ORF from a gene of a HBoV consisting of a polynucleotide encoding the NS2 protein from a HBoV and the NP1 protein from a HBoV, and
with the proviso that the chimeric helper nucleic acid molecule does not comprise further polynucleotides encoding proteins from a HSV.

In another preferred embodiment of the invention, the chimeric helper nucleic acid molecule comprises:
(i) the ORF from a gene of a HSV consisting of a polynucleotide encoding the ICP8 protein from a HSV, preferably from HSV1;
(ii) the ORF from a gene of a HBoV consisting of a polynucleotide encoding the NP1 protein from a HBoV, preferably from HBoV1;
(iii) the VA-RNA polynucleotide from an Ad, preferably from Ad5;
(iv) the E2A gene from an Ad, preferably from Ad5;
(v) the E4 gene, in particular the E4orf6 sequence, from an Ad, preferably from Ad5;
wherein each of the (i) (ii), (iii), (iv) and (v) polynucleotides are inserted as individual expression cassettes within the chimeric helper nucleic acid molecule, and wherein polynucleotides of (i) to (v) are arranged in any order and/or orientation with respect to one another; preferably wherein each of the (i) (ii), (iii), (iv) and (v) polynucleotides are inserted as individual expression cassettes within the chimeric helper nucleic acid molecule, wherein the polynucleotides in the chimeric nucleic acid molecule have the following order from the 5′ to 3′ direction: (i), (ii), (iii), (iv) and (v), and wherein the nucleic acids (i) and (ii) are under the control of a non-endogenous promoter, in particular a different promoter;
with the proviso that the chimeric helper nucleic acid molecule does not comprise (ii) an ORF from a gene of a HBoV consisting of a polynucleotide encoding the NS2 protein from a HBoV and the NP1 protein from a HBoV, and
with the proviso that the chimeric helper nucleic acid molecule does not comprise further polynucleotides encoding proteins from a HSV.

In another preferred embodiment of the invention, the chimeric helper nucleic acid molecule comprises:
(i) the ORF from a gene of a HSV consisting of a polynucleotide encoding the UL12 protein from a HSV and the ICP8 protein from a HSV, preferably from HSV1;
(ii) the ORF from a gene of a HBoV consisting of a polynucleotide encoding the NS2 protein from a HBoV, preferably from HBoV1;
(iii) the VA-RNA polynucleotide from an Ad, preferably from Ad5;
(iv) the E2A gene from an Ad, preferably from Ad5;
(v) the E4 gene, in particular the E4orf6 sequence, from an Ad, preferably from Ad5;
wherein each of the (i) (ii), (iii), (iv) and (v) polynucleotides are inserted as individual expression cassettes within the chimeric helper nucleic acid molecule, and wherein polynucleotides of (i) to (v) are arranged in any order and/or orientation with respect to one another; preferably wherein each of the (i) (ii), (iii), (iv) and (v) polynucleotides are inserted as individual expression cassettes within the chimeric helper nucleic acid molecule, wherein the polynucleotides in the chimeric nucleic acid molecule have the following order from the 5′ to 3′ direction: (i), (ii), (iii), (iv) and (v), and wherein the nucleic acids (i) and (ii) are under the control of a non-endogenous promoter, in particular a different promoter;
with the proviso that the chimeric helper nucleic acid molecule does not comprise (ii) an ORF from a gene of a HBoV consisting of a polynucleotide encoding the NS2 protein from a HBoV and the NP1 protein from a HBoV, and
with the proviso that the chimeric helper nucleic acid molecule does not comprise further polynucleotides encoding proteins from a HSV.

In another preferred embodiment of the invention, the chimeric helper nucleic acid molecule comprises:
(i) the ORF from a gene of a HSV consisting of a polynucleotide encoding the UL12 protein from a HSV and the ICP8 protein from a HSV, preferably from HSV1;
(ii) the ORF from a gene of a HBoV consisting of a polynucleotide encoding the NP1 protein from a HBoV, preferably from HBoV1;
(iii) the VA-RNA polynucleotide from an Ad, preferably from Ad5;
(iv) the E2A gene from an Ad, preferably from Ad5;
(v) the E4 gene, in particular the E4orf6 sequence, from an Ad, preferably from Ad5;
wherein each of the (i) (ii), (iii), (iv) and (v) polynucleotides are inserted as individual expression cassettes within the chimeric helper nucleic acid molecule, and wherein polynucleotides of (i) to (v) are arranged in any order and/or orientation with respect to one another; preferably wherein each of the (i) (ii), (iii), (iv) and (v) polynucleotides are inserted as individual expression cassettes within the chimeric helper nucleic acid molecule, wherein the polynucleotides in the chimeric nucleic acid molecule have the following order from the 5′ to 3′ direction: (i), (ii), (iii), (iv) and (v), and wherein the nucleic acids (i) and (ii) are under the control of a non-endogenous promoter, in particular a different promoter;
with the proviso that the chimeric helper nucleic acid molecule does not comprise (ii) an ORF from a gene of a HBoV consisting of a polynucleotide encoding the NS2 protein from a HBoV and the NP1 protein from a HBoV, and
with the proviso that the chimeric helper nucleic acid molecule does not comprise further polynucleotides encoding proteins from a HSV.

The location, the order and/or the orientation of the functional sequences in the chimeric helper nucleic acid molecule are for example illustrated in FIG. 10 (plasmids Ad-HBoV-HSV and Ad-HBoV-HSV5 according to the invention).

The present invention also relates to a packaging system, comprising:
(a) an AAV transfer nucleic acid molecule, in particular a plasmid (pTransfer) comprising a transgene of interest comprising an Open Reading Frame (ORF) under the control of transcription and translation regulatory elements, wherein the transgene of interest is flanked by inverted terminal repeats (ITR) from an AAV, in particular AAV of serotype 2 (AAV2), AAV of serotype 5 (AAV5), AAV of serotype 8 (AAV8), or AAV of serotype 9 (AAV9), preferably AAV2, AAV5 or AAV9, more preferably AAV2;
(b) a Rep/Cap nucleic acid molecule, in particular a plasmid (pPackaging or pRC or pAAVRep/Cap) providing AAV viral functions and comprising a replication (Rep) gene from AAV2 and a capsid (Cap) gene from an AAV, preferably from an AAV of serotype 2, 5, 8 or 9; and
(c) the chimeric helper nucleic acid molecule according to the invention;
wherein the nucleic acid molecule (a), (b) and (c) are provided as one molecule, two different molecules or three different molecules, preferably three different molecules consisting of or comprising (a), (b) and (c) respectively.

As defined herein, the term “packaging system” refers to a system such as a plasmid comprising structural and packaging genes.

As defined herein, the term “inverted terminal repeats (ITR) from an AAV” refers to regions found at each end of the AAV genome which function together in cis as origins of DNA replication and as packaging signals for the viral genome.

As defined herein, the term “transgene” refers to the gene that will be delivered by the AAV. Examples of transgenes of interest include, but are not limited to, genes or ORFs of therapeutic interest.

In a particular embodiment of the invention, AAV is an AAV of serotype 1, 2, 3, 4, 5, 6, 7, 8, 9, 10, 11, 12 or 13, preferably an AAV of serotype 2, 5, 8 or 9, more preferably an AAV of serotype 2, 5 or 9, even more preferably an AAV of serotype 2.

The present invention also relates to a recombinant adeno-associated virus (rAAV) producer cell transformed with, especially transfected with, the packaging system according to the invention, in particular which is a recombinant E1 complementary producer cell.

In a particular embodiment of the invention, the rAAV producer cell is a mammalian cell, preferably a human cell, more preferably a HEK-293 cell, even more preferably a HEK-293T cell, in particular a recombinant E1 complementary producer cell.

The present invention also relates to a helper-free virus method of producing an infectious recombinant adeno-associated virus (rAAV) in a producing cell line, comprising the steps of:
transforming, in particular transfecting, cells or a cell line, preferably mammalian cells or insect cells, with the packaging system according to the invention, in particular stably transfecting said cell with the packaging system according to the invention, and allowing transfected cells to produce rAAV virions;
harvesting the transformed, in particular transfected cells and lysing them to recover rAAV virions; and
collecting rAAV virions in cell lysates or supernatants and optionally purifying the rAAV virions.

In a particular embodiment of the invention, said helper-free virus method comprises a step of purifying the rAAV virions.

As defined herein, the term “AAV virions” refers to a complete virus particle, for example a wild-type AAV virus particle comprising single-stranded genome DNA packaged into AAV capsid proteins.

As defined herein, the term “rAAV virions” refers to recombinant AAV virions, i.e., an infectious, replication-defective virus composed of an AAV protein shell, encapsidating a DNA molecule of interest which is flanked on both sides by AAV ITRs.

In a particular embodiment of the invention, the producing cell line is selected from the group consisting of HEK-293 cell line, for example HEK-293T cell line, VPC 2.0 cell line, Expi293F cell line and an adherent cell line, and wherein the cells are grown in suspension.

As defined herein, the term “an adherent cell line” refers to cell lines that need solid support for growth, and thus are anchorage-dependent. Examples of adherent cells include, but are not limited to, MRC-5 cells, Hela cells, Vero cells, NIH-3T3 cells, L293 cells, CHO cells, BHK-21 cells, MCF-7 cells, A549 cells, COS cells, HEK 293 cells, Hep G2 cells, SNN-BE(2) cells, BAE-1 cells and SH-SY5Y cells.

In a particular embodiment of the invention, transfection is carried out by chemical transfection, electroporation or sonoporation.

The present invention also relates to a method of producing a replication defective infectious recombinant adeno-associated virus (rAAV) vector particle, comprising the steps of:
transforming, in particular transfecting a cell, preferably a mammalian cell or an insect cell, with the packaging system according to the invention, in particular stably transfecting said cell with the packaging system according to the invention, and allowing transformed, in particular transfected cells to produce rAAV vector particles;
harvesting the transformed, in particular transfected cells and lysing them to recover rAAV vector particles; and
collecting rAAV vector particles in cell lysates or supernatants and optionally purifying the rAAV vector particles.

In a particular embodiment of the invention, the method of producing a replication defective infectious rAAV vector particle comprises a step of purifying the rAAV vector particles.

The present invention also relates to in vitro use of the rAAV producer cell according to the invention, in the production of a replication defective infectious rAAV vector particle.

Other features and advantages of the invention will be apparent from the examples which follow and will also be illustrated in the figures.

### BRIEF DESCRIPTION OF THE FIGURES

FIG. 1. Overview of the different Helper plasmids used in this study. The overall architectures of the plasmids are depicted as schemes containing the genes coming from AAV Helper viruses (light grey=Adenovirus, dark grey=Bocavirus and white=Herpesvirus). The name and size of each plasmid is shown on the left. The use of heterologous promoters is shown with an arrow (pCMV=promoter of the cytomegalovirus and pEFS=core promoter of the eukaryotic translation elongation factor 1α). pALD-X80 and pHelper ALD were purchased from Aldevron, pHelper CB was purchased from Cellbiolabs. All other plasmids were constructed for the purpose of this study by E-zyvec proprietary technology, as described in patent EP3256583B1.

FIG. 2. Determination of the optimal plasmid ratio for the production of rAAV2 in suspension HEK-293T. rAAV2 vectors expressing eGFP reporter gene were produced in HEK-293T cells grown in suspension. Cells were transfected by 3 plasmids (pRC2 vector expressing Rep and Cap, pAAV-GFP control vector expressing GFP under the control of a CMV promoter flanked by AAV2 ITRs and a Helper vector (pALD-X80) expressing at least Adeno E2A, Adeno E4 and Adeno VA helper factors) with FectoVIR-AAV at ratio 1:1 μg DNA/μL reagent. rAAV titers (transducing unit, TU/mL) were determined 72 hours post-transfection by transduction of H1080 cells and by quantification of eGFP+ cells by flow cytometry. The results are expressed as relative rAAV2 transducing units/mL (TU/mL). Different mass ratios were used for this plasmid combination to determine the optimal ratio between these plasmids to generate the highest infectious titer. The resulting copy number for each plasmid was then used for further experiments to keep the copy number of the plasmids equivalent in each condition.

FIG. 3. Comparison of different Helper plasmids for their efficiency to produce rAAV2. rAAV2 vectors expressing eGFP reporter gene were produced in HEK-293T cells grown in suspension. Cells were transfected by 3 plasmids (pRC2 vector expressing Rep and Cap, pAAV-GFP control vector expressing GFP under the control of a CMV promoter flanked by AAV2 ITRs and a Helper vector expressing at least Adeno E2A, Adeno E4 and Adeno VA helper factors) with FectoVIR-AAV at ratio 1:1 μg DNA/μL reagent. AAV titers (transducing unit, TU/mL) were determined 72 hours post-transfection by transduction of H1080 cells and by quantification of eGFP+ cells by flow cytometry. The results are expressed as relative rAAV2 transducing units/mL (TU/mL).

FIG. 4. Addition of genes from other Helper viruses on the Helper plasmid and impact on rAAV2 production. rAAV2 vectors expressing eGFP reporter gene were produced in HEK-293T cells grown in suspension. Cells were transfected by 3 plasmids (pRC2 vector expressing Rep and Cap, pAAV-GFP control vector expressing GFP under the control of a CMV promoter flanked by AAV2 ITRs and a Helper vector expressing at least Adeno E2A, Adeno E4 and Adeno VA helper factors) with FectoVIR-AAV at ratio 1:1 μg DNA/μL reagent. AAV titers (transducing unit, TU/mL) were determined 72 hours post-transfection by transduction of H1080 cells and by quantification of eGFP+ cells by flow cytometry. The results are expressed as relative rAAV2 transducing units/mL (TU/mL) normalized to the infectivity of rAAV produced with pALD-X80 (which contains Adenovirus-only genes) and represented in fold increase for Fig. A, B and F. A) Infectivity of rAAV produced with plasmids carrying different Helper genes coming either from Bocavirus (Ad-HBoV1 and Ad-HBoV2 in grey) or Herpesvirus (Ad-HSV1 and Ad-HSV2 in white) were added to the Adenovirus genes, generating double chimer Helper plasmids. B) Infectivity of rAAV produced with a plasmid carrying genes from Adenovirus, Bocavirus and Herpesvirus (triple chimer plasmid). C) Quantification of Genome Copies per mL (GC/mL) D) Viral Particles per mL (VP/mL) E) full/empty ratio (GC/VP) F) TU/VP of rAAV2 produced with either pALD-X80 or the triple chimeric plasmid.

FIG. 5. Evaluation of the versatility and efficiency of the triple chimeric Helper plasmid to produce rAAV2 in various packaging cell lines derived from HEK-293. rAAV2 vectors expressing eGFP reporter gene were produced in A) VPC 2.0 cells grown in suspension in Viral Production Medium, or B) 293-F cells grown in suspension in Freestyle™ 293 medium or C) Expi293F™ cells grown in suspension in Expi293™ Expression medium. Cells were transfected by 3 plasmids (pRC2 vector expressing Rep and Cap, pAAV-GFP control vector expressing GFP under the control of a CMV promoter flanked by AAV2 ITRs and a Helper vector expressing at least Adeno E2A, Adeno E4 and Adeno VA helper factors) with FectoVIR-AAV at ratio 1:1 μg DNA/μL reagent. AAV titers (transducing unit, TU/mL) were determined 72 hours post-transfection by transduction of H1080 cells and by quantification of eGFP+ cells by flow cytometry. The results are expressed as relative rAAV2 transducing units/mL (TU/mL) normalized to the infectivity of rAAV produced with pALD-X80 and represented in fold increase.

FIG. 6. Evaluation of the versatility and efficiency of the triple chimeric Helper plasmid to produce rAAV2 using different transfection reagents. rAAV2 vectors expressing eGFP reporter gene were produced in HEK-293T cells grown in suspension. Cells were transfected by 3 plasmids (pRC2 vector expressing Rep and Cap, pAAV-GFP control vector expressing GFP under the control of a CMV promoter flanked by AAV2 ITRs and a Helper vector expressing at least Adeno E2A, Adeno E4 and Adeno VA helper factors) with either FectoVIR-AAV or PEIpro or TransIT-VirusGEN at ratio 1:1 μg DNA/μL reagent. AAV titers (transducing unit, TU/mL) were determined 72 hours post-transfection by transduction of H1080 cells and by quantification of eGFP+ cells by flow cytometry. The results are expressed as relative rAAV2 transducing units/mL (TU/mL) normalized to the infectivity of rAAV produced with pALD-X80 and represented in fold increase.

FIG. 7. Evaluation of the versatility and efficiency of the triple chimeric Helper plasmid to produce rAAV of different serotypes. A) rAAV5, or B) rAAV8, or C) rAAV9 vectors expressing eGFP reporter gene were produced in HEK-293T cells grown in suspension. Cells were transfected by 3 plasmids (pRC5 or pRC8, or pRC9 vector expressing Rep and serotype specific Cap, pAAV-GFP control vector expressing GFP under the control of a CMV promoter flanked by AAV2 ITRs and a Helper vector expressing at least Adeno E2A, Adeno E4 and Adeno VA helper factors) with FectoVIR-AAV at ratio 1:1 μg DNA/μL reagent. AAV titers (transducing unit, TU/mL) were determined 72 hours post-transfection by transduction of H1080 cells and by quantification of eGFP+ cells by flow cytometry. The results are expressed as relative rAAV2 transducing units/mL (TU/mL) normalized to the infectivity of rAAV produced with pALD-X80 and represented in fold increase.

FIG. 8. A) pALD-X80 (18876 bp) by Aldevron; B) pALD-HELP (11584 bp) by Aldevron and C) VPK-402 (11635 bp) by Cellbiolabs.

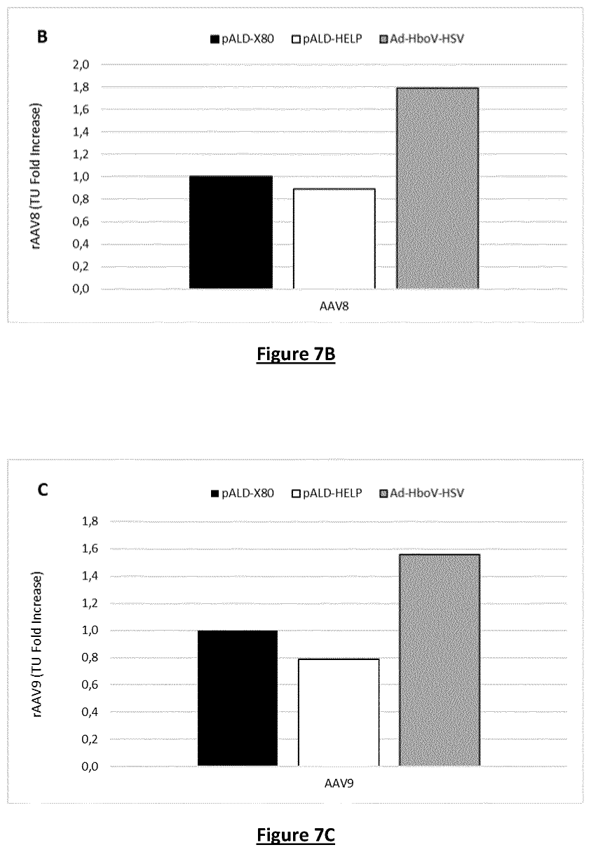

FIG. 9. Plasmid Ad-HBoV-HSV (13324 bp) according to the invention.

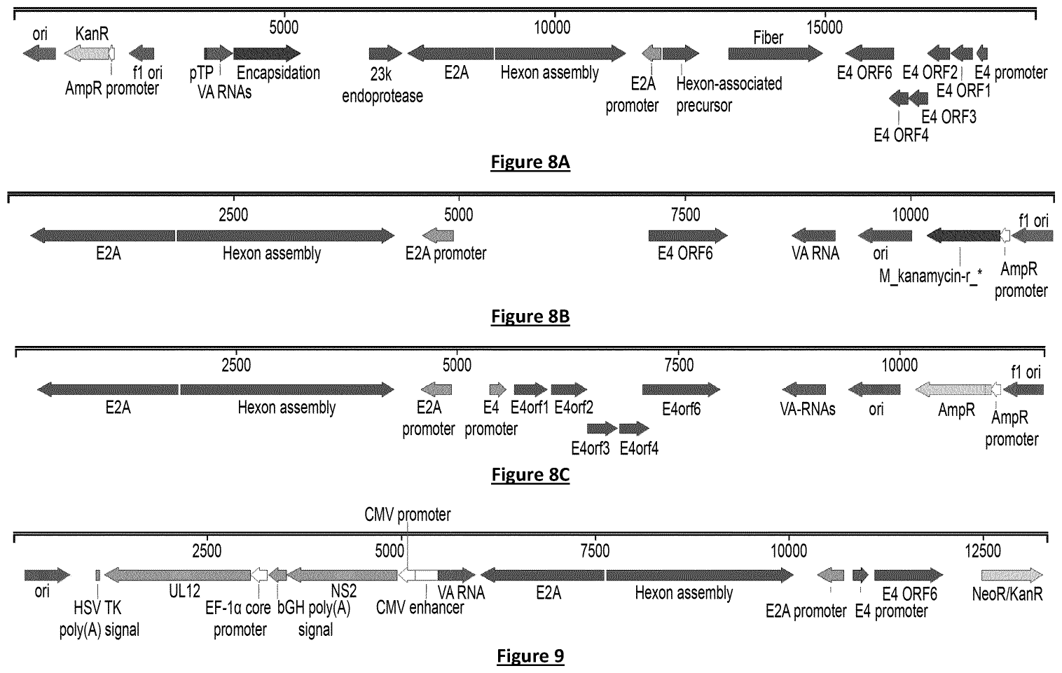

FIG. 10. Helper plasmids used for rAAV production: Plasmid Ad-HBoV-HSV (13324 bp) of SEQ ID NO:18 according to the invention, Plasmid Ad-HBoV-HSV5 (15034 bp) of SEQ ID NO:35 according to the invention, and comparative Plasmid Ad-HBoV5 (11651 bp) of SEQ ID NO:36 as disclosed in Wang et al., Mol. Ther. 2018, 11, 40-51. All 3 plasmids display the same architecture regarding Adenoviral elements (VA-RNA, E2A and E4orf6). Plasmid Ad-HBoV-HSV further comprises a polynucleotide encoding the UL12 protein from HSV1 and a polynucleotide encoding the NS2 protein from HBoV1. Plasmid Ad-HBoV-HSV5 further comprises a polynucleotide encoding the ICP8 protein from HSV1 and a polynucleotide encoding the NS2 protein from HBoV1. Comparative Plasmid Ad-HBoV5 further comprises a polynucleotide encoding the NS2 and NP1 proteins from HBoV1, and a polynucleotide encoding the self-cleaving peptide P2A of SEQ ID NO:34. Said HSV and HBV proteins are driven either by pCMV or pEFS promoters.

FIG. 11. rAAV production efficiency (rAAV VG/mL titers produced in HEK293T). 11A) rAAV2 or 11B) rAAV5 or 11C) rAAV8 or 11D) rAAV9 production efficiency using the following pHelpers: Plasmid Ad-HBoV-HSV and Plasmid Ad-HBoV-HSV5 according to the invention, and comparative Plasmid Ad-HBoV5 as disclosed in Wang et al., Mol. Ther. 2018, 11, 40-51. For an easier representation, the proteins from HBoV and HSV which are present in the pHelpers are shown at the top of the graph.

### EXAMPLES

### Materials and Methods

### Cell Culture

HEK-293T (ATCC® CRL-3216™): Human embryonic kidney cells were grown in suspension, in Freestyle F17 medium, supplemented with 8 mM glutamine, 100 U/mL of penicillin, 100 μg/mL of streptomycin and 0.1% Pluronic. Cells were incubated at 37° C. in a 8% CO₂ in air atmosphere under agitation (130 rpm—orbital of 50 mm).

Freestyle™ 293-F cells (Gibco™ R79007): Human embryonic kidney cells are derived from the HEK-293 parental cell line. Cell were cultivated in suspension in Freestyle™ 293 medium, supplemented with 8 mM L-glutamine, 100 U/mL of penicillin, 100 μg/mL of streptomycin and 0.1% Pluronic. Cells were incubated at 37° C. in a 8% CO₂ in air atmosphere under agitation (130 rpm—orbital of 50 mm).

Viral Production Cells 2.0 (Gibco™ A49784): Human embryonic kidney cells are derived from the HEK-293F parental cell line, itself derived from HEK-293 cell line. Cells were grown in suspension in Viral Production medium, supplemented with 8 mM L-glutamine, 100 U/mL of penicillin and 100 μg/mL of streptomycin. Cells were incubated at 37° C. in a 8% CO₂ in air atmosphere under agitation (130 rpm—orbital of 50 mm).

Expi293F™ (Gibco™ A14527): Human embryonic kidney cells are derived from the HEK-293 parental cell line. Cells were cultivated in suspension in Expi293™ Expression medium, supplemented with 100 U/mL of penicillin and 100 μg/mL of streptomycin. Cells were incubated at 37° C. in a 8% CO₂ in air atmosphere under agitation (130 rpm—orbital of 50 mm).

HT-1080 (ATCC® CCL-121™): Human Fibrosarcoma cells were grown in DMEM 4.5 g/L glucose with 10% FBS supplemented with 2 mM L-glutamine, 100 U/mL of penicillin, 100 μg/mL of streptomycin at 37° C. in a 5% CO₂ in air atmosphere.

CHO-K1 (ATCC° CCL-61™): a cell line derived as a subclone from the parental CHO cell line, which was initiated from a biopsy of an ovary of an adult, female Chinese hamster. Cells were grown in RPMI with 10% FBS supplemented with 2 mM L-glutamine, 100 U/mL of penicillin, 100 μg/mL of streptomycin at 37° C. in a 5% CO₂ in air atmosphere.

### Recombinant Virus Production

HEK-293T (ATCC° CRL-3216™): Human embryonic kidney cell is a highly transfectable derivative of human embryonic kidney 293 cells and contains the SV40 T-antigen. HEK-293T cells are widely used for recombinant virus production, gene expression and protein production.

HEK-293T cells were seeded at 1×10⁶ cells/mL in 28.5 mL of Freestyle F17 supplemented with 8 mM L-glutamine, 100 U/mL of penicillin, 100 μg/mL of streptomycin and 0.1% Pluronic in 125 mL flask Erlenmeyer. Cells were incubated at 37° C. in a 8% CO₂ in air atmosphere under agitation (130 rpm—orbital of 50 mm).

Freestyle™ 293-F cells (Gibco™ R79007): Human embryonic kidney cell is a highly transfectable derivative of human embryonic kidney 293 cells. Freestyle™ 293F cells are part of the Freestyle™ MAX 293 expression system and are used for gene expression and recombinant protein production.

Freestyle™ 293-F cells were seeded at 1×10⁶ cells/mL in 28.5 mL of Freestyle™ 293 supplemented with 8 mM L-glutamine, 100 U/mL of penicillin, 100 μg/mL of streptomycin and 0.1% Pluronic in 125 mL flask Erlenmeyer. Cells were incubated at 37° C. in a 8% CO₂ᵥ in air atmosphere under agitation (130 rpm—orbital of 50 mm).

Viral Production Cells 2.0 (Gibco™ A49784): Human embryonic kidney cells are a clonal cell line derived from the HEK293F parental cell line and a core component of the AAV-MAX Helper-Free AAV Production System. Viral Production Cells 2.0 are highly transfectable, does not contain the SV40 T-antigen and are used for adeno-associated virus (AAV) production.

Viral Production Cells 2.0 cells were seeded at 1×10⁶ cells/mL in 28.5 ml of Viral Production medium, supplemented with 8 mM L-glutamine, 100 U/mL of penicillin and 100 μg/mL of streptomycin in 125 mL flask Erlenmeyer. Cells were incubated at 37° C. in a 8% CO₂ in air atmosphere under agitation (130 rpm—orbital of 50 mm).

Expi293F™ (Gibco™ A14527): Human embryonic kidney cells are derived from the HEK-293 parental cell line. Expi293FT are highly transfectable and are mostly used for recombinant protein production and for recombinant virus production.

Expi293F™ cells were seeded at 1×10⁶ cells/mL in 28.5 mL of Expi293™ Expression medium, supplemented with 100 U/mL of penicillin and 100 μg/mL of streptomycin in 125 mL flask Erlenmeyer. Cells were incubated at 37° C. in a 8% CO₂ in air atmosphere under agitation (130 rpm—orbital of 50 mm).

Recombinant Adeno Associated Viruses (rAAVs) were produced in HEK-293T, VPC 2.0 or Expi293F™ cells, seeded at 1×10⁶ cells/mL and cultivated for 24 h at 37° C., 8% CO₂ before being co-transfected with 3 plasmids, a pRC vector expressing Rep and Cap, the pHelper vector expressing at least Adeno E2A, Adeno E4 and Adeno VA helper factors, and pAAV-GFP (catalog number AAV-400, Cell BIOLABS, INC.) control vector expression the GFP under control of a CMV promoter. Plasmids were diluted (Standard: pAAV-GFP 7.23×10⁴ copies/cell—pRC 5.32×10⁴ copies/cell—pHelper 1×10⁴ copies/cell) in 1.5 mL of non-supplemented culture medium, then FectoVIR-AAV was added onto the diluted DNA (ratio 1 μL per μg of total DNA), mixed with a vortex, and incubated for 30 minutes at room temperature. Transfection complexes were added onto the cells and the Erlenmeyer flask was incubated for 72 h at 37° C. in a 8% CO₂ in air atmosphere under agitation (130 rpm—orbital of 50 mm).

Transfected cells were harvested 3 days after transfection and centrifugated 5 minutes at 1000 rpm, the supernatant was discarded, and the pellet was resuspended in 2 mL of PBS. Cells were then lysed to liberate rAAVs using 3 successive freeze/thaw cycles at −80° C. and 37° C. Then, 2 mL of the lysate were collected and centrifugated 30 minutes at 14000 rpm to separate rAAVs from cell debris. Supernatant containing rAAVs were then analyzed.

The transducing unit titer (TU/mL) was determined by using recombinant Adeno Associated Viruses expressing the GFP reporter gene after infection of permissive cells, HT-1080 or CHO-K1, depending on the produced rAAV serotype, in 96-well plate. Briefly, permissive cells were seeded at 7×10³ cells per well and incubated 4 hours at 37° C. in a 5% CO₂ in air atmosphere. Harvested rAAVs were then serial diluted in supplemented culture medium and added on permissive cells as a replacement of the previous culture medium used. The GFP expression was analyzed by flow cytometry 72 h after transduction to determine the transducing units.

The capsid titer (VP/mL) was determined by using AAV Titration ELISA kit (catalog number PRAAV2/PRAAV5/PRAAV8/PRAAV9, PROGEN INC), following the manufacturer recommendations and protocol and according to the serotype of the recombinant Adeno Associated Viruses produced and analyzed.

The genome titer (VG/mL) was determined by qPCR (QuantStudio™3—ThermoFisher). Plasmid DNA coding for GFP was used from a concentration of 2×10⁸ copies/μL to 2×10² copies/μL to generate a standard curve using primers (Qiagen) targeting GFP reporter gene expressed by the rAAV produced, and the SensiFAST probe Lo-ROX kit (Ozyme).

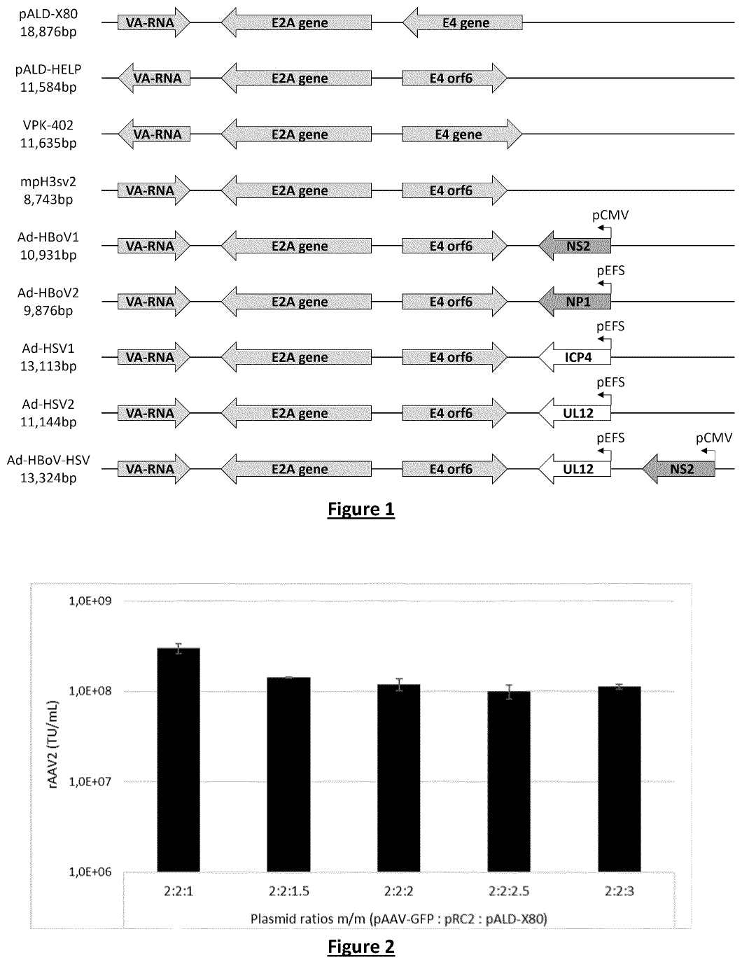

FIG. 1 discloses an overview of all helper plasmid maps used in this study. All the helper plasmids were constructed with at least the minimal Ad-helper elements (VA-RNA, E2A and E4). Additional elements from other helper viruses (HBoV and HSV) were also added to compare their potency to produce rAAV compared to the commercial helper plasmids.

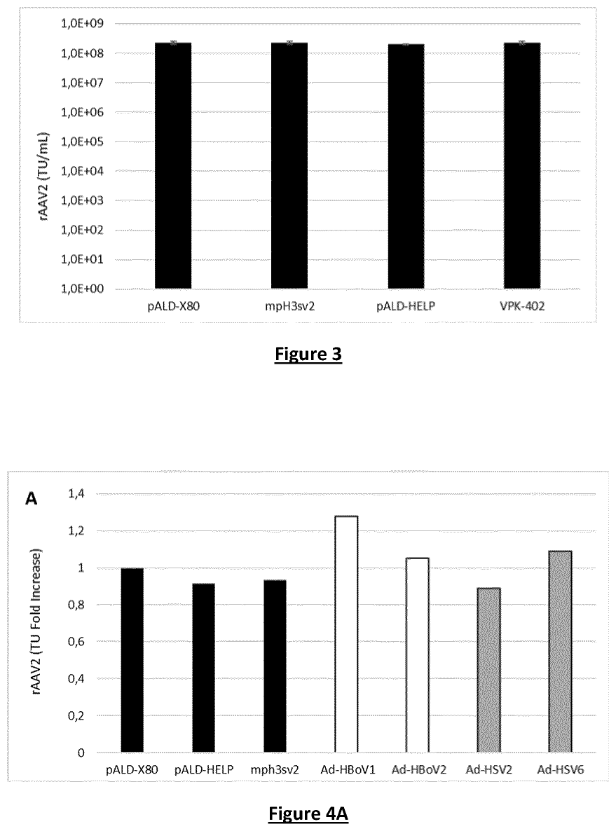

FIG. 2 discloses viral titer (transducing units per mL) of rAAV2 produced after transfection of HEK 293t cells using various ratios of pHelper (pALD-X80). The mass ratio of 2:2:1 (pTransgene:pRC:pHelper) gave the best viral titer compared to the other tested ratios and was thus selected for further experiments to compare the different pHelpers showed in this study.

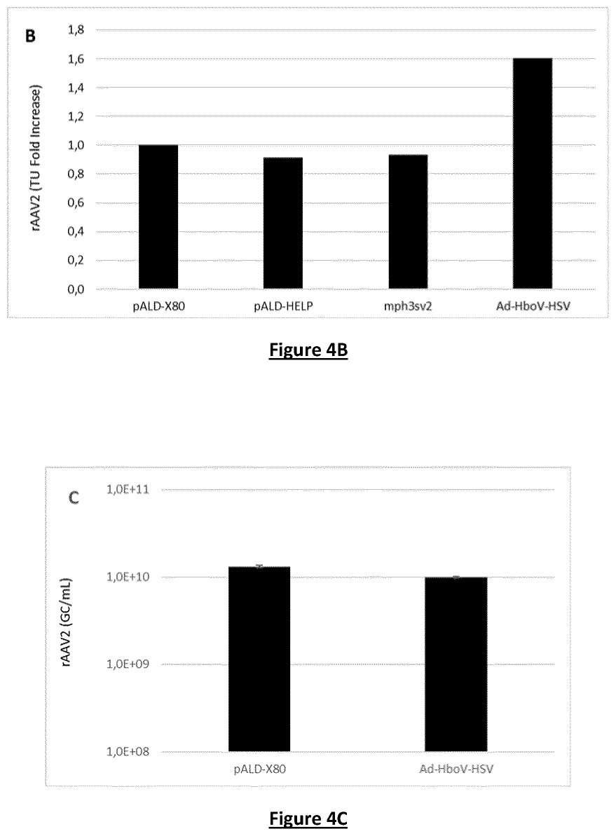

FIG. 3 discloses viral titer (transducing units per mL) of rAAV2 produced after transfection of HEK 293t cells using various pHelpers. Three commercial helper plasmids were used as reference (pALD-X80 and pALD-HELP from Aldevron and VPK-402 from Cellbiolabs) to compare with a minimal Adenovirus-only elements pHelper (mpH3sV2). The minimal pHelper (mpH3sV2) displayed similar viral titer than the other commercial helper plasmids, suggesting that all the sequences that had been removed in the mpH3sV2 construct were not required to support efficient rAAV production. The mpH3sV2 (˜8 kb) pHelper was thus used as a frame for further optimization of the pHelper.

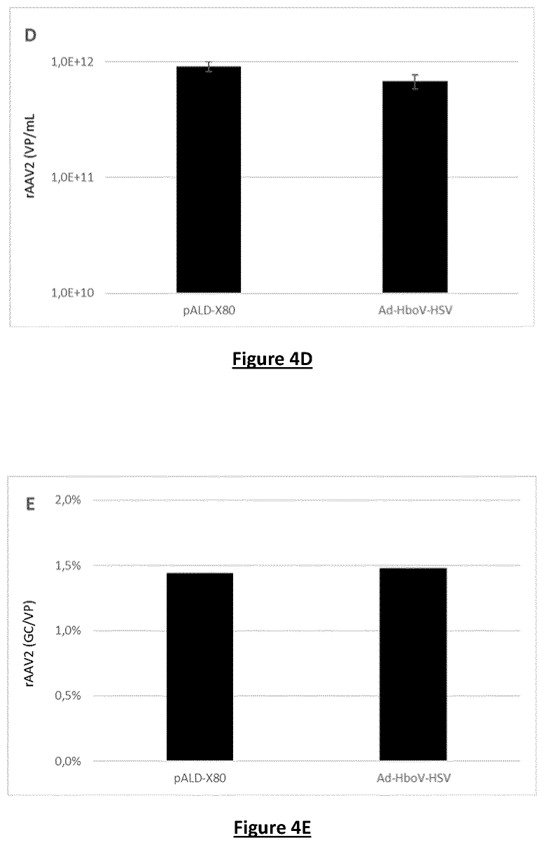

FIG. 4 discloses:

A) Viral titer (transducing units per mL) of rAAV2 produced after transfection of HEK 293t cells using various pHelpers containing either Ad-only elements (pALD-X80, pALD-HELP and mpH3sV2, in black) or Ad and HBoV elements (Ad-HBoV1 and Ad-HBoV2, in white) or Ad and HSV elements (Ad-HSV2 and Ad-HSV6, in grey) (called the double chimeric vectors). The addition of specific helper elements from HBoV and HSV could enhance the viral titer of viruses produced with these pHelpers compared to Ad-only pHelpers, suggesting that the elements introduced in the double chimeric vectors were beneficial to the efficient production of rAAV2 (see Ad-HBoV1 and Ad-HSV6 compared to any black plot).
B) Viral titer (transducing units per mL) of rAAV2 produced after transfection of HEK 293t cells using various pHelpers containing either Ad-only elements (pALD-X80, pALD-HELP and mpH3sV2) or Ad and HBoV and HSV elements (Ad-HBoV-HSV). As seen in FIG. 4A, the addition of either HBoV or HSV elements was beneficial for the efficient production of rAAV2, the inventors have thus assessed the potency of a pHelper containing all Ad, HBoV and HSV elements that were found to be involved in rAAV production to check if it could further improve rAAV2 production. FIG. 4B shows that the combination of elements coming from all three helper viruses (called the triple chimeric vector or triple chimeric pHelper) was better than the double chimeric vector (˜1.6 fold increase compared to 1.2 fold increase respectively when using pALD-X80 as a reference). These results suggested that a combination of helper elements coming from three distinct viruses is so far the best combination the inventors could obtain to efficiently produce infectious rAAV2.

C) GC titer (Genome Copy per mL) of rAAV2 produced with either pALD-X80 or Ad-HBoV-HSV analyzed by qPCR. Since the viruses produced with the triple chimeric pHelper were more infectious than those produced with commercial helper plasmids, the aim of this experiment was to check if this enhanced viral titer was dependent on the amount of encapsidated DNA. The results showed no significant differences between rAAV2 produced with either pALD-X80 or our triple chimeric pHelper.

D) VP titer (Viral Particles per mL) of rAAV2 produced with either pALD-X80 or Ad-HBoV-HSV analyzed by ELISA. Both rAAV2 productions with pALD-X80 and the triple chimeric pHelper displayed similar VP titers, suggesting that the full empty ratio (filled capsids versus empty capsids) of rAAV2 produced with the triple chimeric pHelper was unchanged compared to commercial helper plasmid.

E) Percentage of capsids containing a viral DNA of rAAV2 produced with either pALD-X80 or Ad-HBoV-HSV (GC/VP). As shown in FIGS. 4C and 4D, GC and VP titers were similar between rAAV2 produced with either pALD-X80 or the triple chimeric pHelper, thus the GC/VP ratio (corresponding to the number of total capsids containing a viral DNA copy) were also similar.

F) Overall quality of rAAV2 production (TU/VP) produced with either pALD-X80 or Ad-HBoV-HSV pHelper. Since rAAV2 produced with the triple chimeric pHelper were more infectious than those produced with pALD-X80 (FIG. 4B) but did not display enhanced GC or VP titers (FIGS. 4C and 4D), the quality of the rAAV2 production in terms of ratio of infectious versus non-infectious particles (TU/VP) has been analyzed. As shown in FIG. 4F, this ratio in increased when rAAV2 were produced with the triple chimeric pHelper compared to pALD-X80. This increased ratio suggested that the triple chimeric pHelper enabled the production of better-quality viruses compared to its pALD-X80 counterpart.

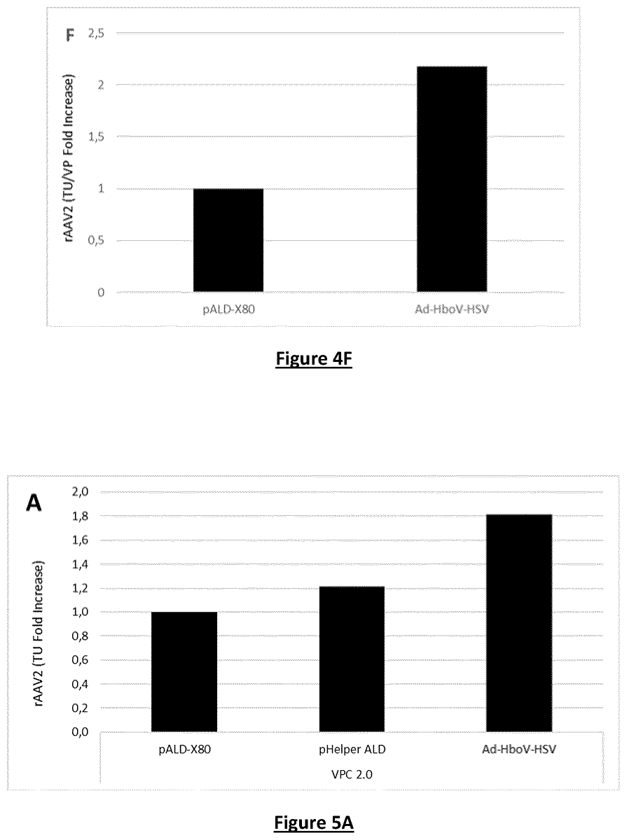

FIG. 5 discloses viral titer (transducing unit/mL) of rAAV2 produced after transfection of various producer cell lines: VPC 2.0 (FIG. 5A), HEK 293F (FIG. 5B) and Expi293F (FIG. 5C) using pHelpers containing Ad-only elements (pALD-X80, pALD-HELP) or Ad+HboV+HSV elements (Ad-HBoV-HSV). Results were normalized to the infectivity of rAAV produced with pALD-X80 and represented in fold increase. Results suggested that the enhancing effects of the combination of Ad, HBoV and HSV elements within a single plasmid, observed previously in HEK293T, were not specific to that cell line and could also be observed in the three producer cell lines tested with a fold increase ranging from ˜1.8 to ˜4.5. FIGS. 5A to 5C show the versatility of the triple chimeric pHelper for different cell lines.

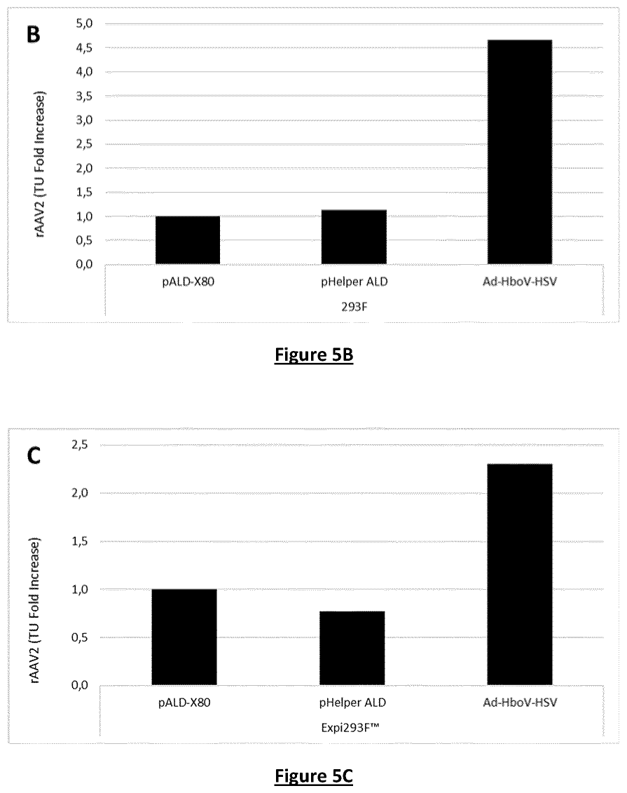

FIG. 6 discloses viral titer (transducing unit/mL) of rAAV2 produced after transfection of HEK 293T using pHelpers containing Ad-only elements or Ad+HboV+HSV elements (Ad-HBoV-HSV). Cells were transfected using three different transfection reagents: FectoVIR-AAV (Polyplus), PEIpro (Polyplus) and TransIT-VirusGEN (Mirus Bio). Results were normalized to the infectivity of rAAV produced with pALD-X80 and represented in fold increase.

Since all the previous results were obtained using FectoVIR-AAV for the transfection of HEK293T, the aim was to check if the increased viral titer obtained with the triple chimeric pHelper (Ad-HBoV-HSV) was related to a specific transfection reagent. Thus, HEK293T cells were transfected using two other transfection reagent: PEIpro and TransIT-VirusGEN.

Titers obtained with the pALD-X80 and those obtained with the triple chimeric pHelper (Ad-HBoV-HSV) using different transfection reagent suggested that the increase observed with the triple chimeric pHelper was not related to the transfection reagent used.

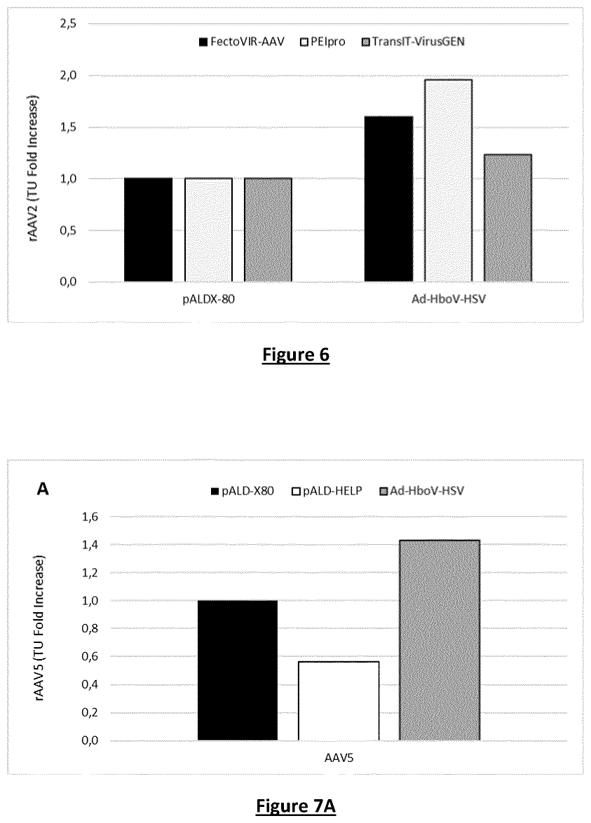

FIG. 7 discloses viral titer (transducing unit/mL) of various serotypes of rAAV: rAAV5 (FIG. 7A), rAAV8 (FIG. 7B) and rAAV9 (FIG. 7C) using pHelpers containing Ad-only elements (pALD-X80, pALD-HELP) or Ad and HboV and HSV elements (Ad-HBoV-HSV). Results were normalized to the infectivity of rAAV produced with pALD-X80 and represented in fold increase. Results suggested that the enhancing effects of the combination of Ad, HBoV and HSV elements within a single plasmid, observed previously for the production of rAAV2, were not specific to that serotype and could also be observed for the production of other rAAV serotypes with a fold increase ranging from ˜1.4 to ˜1.8. FIGS. 7A to 7C show the versatility of the triple chimeric pHelper for different AAV serotypes.

Absence/limitations of unnecessary sequences: Some commercial pHelpers on the market have unnecessary sequences coming from adenovirus genome. To a gene therapy purpose, these sequences should be avoided for a better safety and lower toxicity. To this aim, the inventors have delimited and removed all unnecessary sequences from the adenovirus genome, to only keep the elements that are involved in rAAV production.

Unnecessary sequences refer to the sequences that are not involved in rAAV production, i.e., sequences usually flanking required genes present in commercial plasmids due to technical cloning limitations. Any nucleotide/sequence other than those found in genes of interest (from promoter to poly Adenylation signal) were thus excluded from the plasmid of the invention.

Increased titers: One of the major concerns of rAAV production was to increase viral titer to meet the requirement of large amount of infectious viruses required for rAAV-based gene therapy. The plasmid of the invention led to an increased proportion of infectious viruses compared to other commercial Helper plasmids (up to ˜2-fold increase depending on AAV serotype).

Quality of the viruses: The plasmid of the invention led to an increased number of infectious viruses compared to the reference commercial pHelpers. Also, the inventors showed that a viral production done with the plasmid of the invention had an improved infectious titer per total capsid (TU/VP) suggesting that the rAAV produced with the plasmid of the invention were of better quality (more infectious). Considering that current viral production methods lead to a very low quality of virus batchs (most of the viruses produced are non-infectious), finding ways to improve the infectiousness of the rAAV could improve the overall efficacy of the rAAV-based gene therapy while lowering the immune response targeting these non-infectious viruses.

Versatile pHelper for different AAV serotypes: The Helper plasmid of the invention has been tested on various AAV serotypes to assess its efficiency regarding the current methods used for production of serotypes of interest, i.e, serotypes 2, 5, 8 and 9. The plasmid of the invention displayed increased infectious titers on all serotypes mentioned above compared to the commercial Helper plasmids. The increase extent was serotype-dependant, ranging from 2-fold for AAV2 and up to 3-fold for AAV5, AAV8 and AAV9, in particular from 1.5-fold to 2-fold for AAV2, AAV5, AAV8 or AAV9.

Versatile pHelper for different cell lines: The Helper plasmid of the invention was tested in several mammalian cell lines derived from HEK-293, i.e., HEK-293T, HEK-293F, VPC 2.0 and Expi293F cells. Among the four cell lines tested, the Helper plasmid of the invention showed increased infectious titer compared to the commercial Helper plasmid, suggesting that the plasmid of the invention works on different cell cultures conditions depending on the needs.

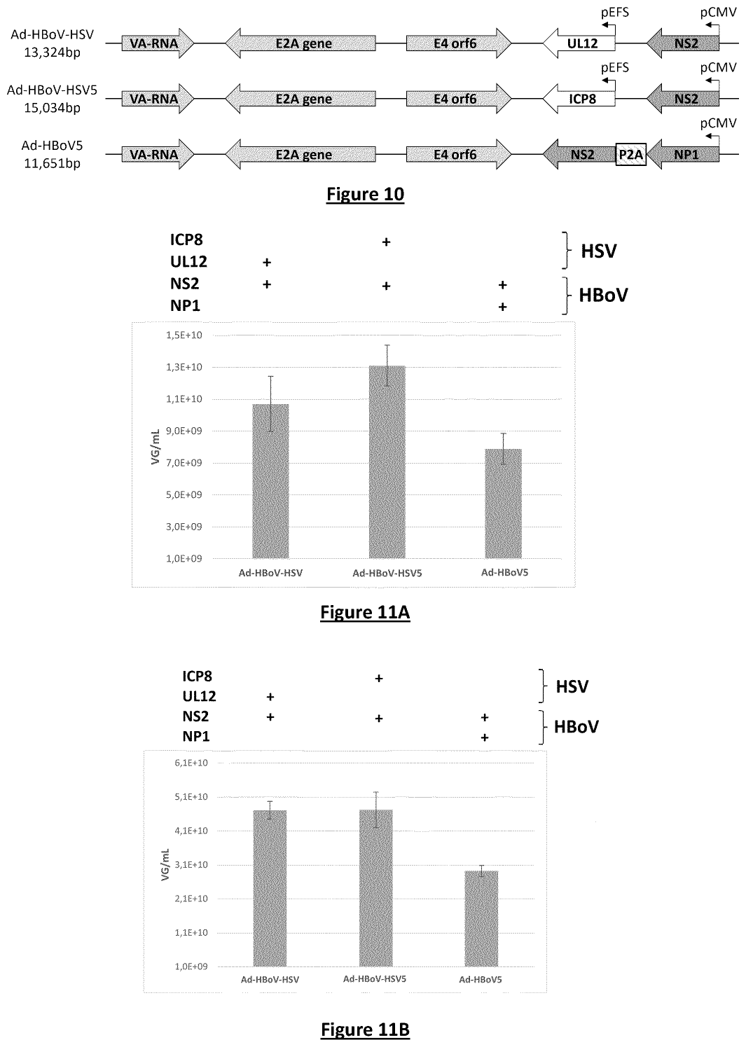

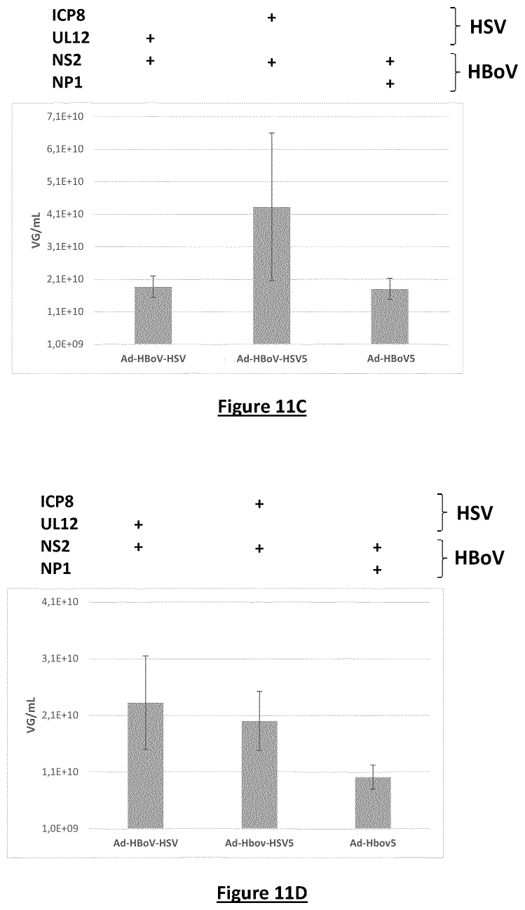

The inventors have compared rAAV production efficiency between triple chimeric pHelpers according to the invention (Plasmid Ad-HBoV-HSV and Plasmid Ad-HBoV-HSV5) and the comparative Plasmid Ad-HBoV5 disclosed in Wang et al., Mol. Ther. 2018, 11, 40-51. The triple chimeric pHelpers according to the invention led to a high efficient production of a plurality of rAAV serotypes (2, 5, 8 and 9) (FIGS. 10 and 11).

While the invention has been described in terms of various preferred embodiments, the skilled person will appreciate that various modifications, substitutions, omissions and changes may be made without departing from the scope thereof. Accordingly, it is intended that the scope of the present invention be limited by the scope of the following claims, including equivalents thereof.

1. A chimeric helper nucleic acid molecule suitable for use in a method of producing infectious recombinant adeno-associated virus (rAAV) comprising:
(i) an ORF from a gene of a HSV consisting of a polynucleotide encoding at least one protein of a HSV, comprising a polynucleotide encoding the UL12 protein from a HSV or the ICP8 protein from a HSV;
(ii) an ORF from a gene of a HBoV consisting of a polynucleotide encoding the NS2 protein from a HBoV or the NP1 protein from a HBoV;
(iii) the VA-RNA polynucleotide from an Ad;
(iv) the E2A gene from an Ad;
(v) the E4 gene, in particular the E4orf6 sequence, from an Ad;
wherein each of the (i) (ii), (iii), (iv) and (v) polynucleotides are inserted as individual expression cassettes within the chimeric helper nucleic acid molecule, and wherein polynucleotides of (i) to (v) are arranged in any order and/or orientation with respect to one another;
with the proviso that the chimeric helper nucleic acid molecule does not comprise (ii) an ORF from a gene of a HBoV consisting of a polynucleotide encoding the NS2 protein from a HBoV and the NP1 protein from a HBoV.

2. The chimeric helper nucleic acid molecule according to claim 1, wherein the ORF from a gene of a HSV consists of a polynucleotide encoding the UL12 protein from a HSV.

3. The chimeric helper nucleic acid molecule according to claim 1, wherein the ORF from a gene of a HBoV consists of a polynucleotide encoding the NS2 protein from a HBoV.

4. The chimeric helper nucleic acid molecule according to claim 1, wherein the polynucleotides in the chimeric nucleic acid molecule have the following order from the 5′ to 3′ direction: (i), (ii), (iii), (iv) and (v), and wherein the nucleic acids (i) and (ii) are under the control of a non-endogenous promoter.

5. The chimeric helper nucleic acid molecule according to claim 1, which further comprises additional adenovirus genes.

6. The chimeric helper nucleic acid molecule according to claim 1, further comprising:
an origin of replication (ori); and/or
a herpes simplex virus thymidine kinase (HSV TK polyA) signal downstream from the HSV UL12 ORF; and/or
a bovine growth hormone polyadenylation (bGH polyA) signal downstream from the HBoV NS2 ORF; and/or
a transcription regulation element such as a cytomegalovirus (CMV) promoter, a CMV enhancer, an EFS promoter, a SV40 virus promoter, a CBA promoter, a CAG promoter, an E2A promoter and/or an E4 promoter to control the transcription of the HSV and HBoV ORFs; and/or
a nucleic acid encoding a marker protein such as the kanamycin resistance gene.

7. The chimeric helper nucleic acid molecule according to claim 1, wherein:
the polynucleotide encoding the UL12 protein from HSV1 is of SEQ ID NO:1;
the polynucleotide encoding the NS2 protein from HBoV1 is of SEQ ID NO:2;
the VA-RNA polynucleotide from Ad5 is of SEQ ID NO:3;
the E2A gene from Ad5 is of SEQ ID NO:4;
the E4 gene from Ad5 is of SEQ ID NO:5; and/or
the polynucleotide encoding the ICP8 protein from HSV1 is of SEQ ID NO:32.

8. The chimeric helper nucleic acid molecule according to claim 1, wherein:
the ori is a nucleotide sequence of SEQ ID NO:7;
the HSV TK polyA signal is a polynucleotide of sequence SEQ ID NO:8;
the bGH poly A signal is a polynucleotide of sequence SEQ ID NO:9;
the CMV promoter is a polynucleotide of sequence SEQ ID NO:10;
the CMV enhancer is a polynucleotide of sequence SEQ ID NO:11;
the EFS promoter is a polynucleotide of sequence SEQ ID NO:12;
the E2A promoter is a polynucleotide of sequence SEQ ID NO:13;
the E4 promoter is a polynucleotide of sequence SEQ ID NO:14;
the kanamycin resistance gene is of sequence SEQ ID NO:15;
the amino acid sequence of the UL12 protein from HSV1 is of SEQ ID NO:19;
the amino acid sequence of the NS2 protein from HBoV1 is of SEQ ID NO:20;
the amino acid sequence of the E2A protein from Ad5 is of SEQ ID NO:21;
the amino acid sequence of the E4 protein from Ad5 is of SEQ ID NO:22, SEQ ID NO:29, SEQ ID NO:30 or SEQ ID NO:31;
the amino acid sequence of the E4orf6 sequence from Ad5 is of SEQ ID NO:23;
the nucleotide sequence encoding the NP1 protein from HBoV1 is of SEQ ID NO:24;
the amino acid sequence of the NP1 protein from HBoV1 is of SEQ ID NO:25;
the El gene from Ad5 is a polynucleotide of SEQ ID NO:26;
the amino acid sequence of the El protein from Ad5 is of SEQ ID NO:27 or SEQ ID NO:28; and/or
the amino acid sequence of the ICP8 protein from HSV1 is of SEQ ID NO:33.

9. The chimeric helper nucleic acid molecule according to claim 1, comprising (i) a polynucleotide insert comprising the EFS promoter, the polynucleotide encoding the UL12 protein from HSV1 and the HSV TK polyA signal, whose sequence is the sequence of SEQ ID NO:16, and (ii) a polynucleotide insert comprising the CMV promoter, the polynucleotide encoding the NS2 protein from HBoV1 and the bGH polyA signal, whose sequence is the sequence of SEQ ID NO:17.

10. The chimeric helper nucleic acid molecule according to claim 1, which is a plasmid, in particular is the plasmid Ad-HBoV-HSV of SEQ ID NO:18, or the plasmid Ad-HBoV-HSV5 of SEQ ID NO:35.

11. A packaging system, comprising:
(a) an AAV transfer nucleic acid molecule comprising a plasmid (pTransfer) comprising a transgene of interest comprising an Open Reading Frame (ORF) under the control of transcription and translation regulatory elements, wherein the transgene of interest is flanked by inverted terminal repeats (ITR) from an AAV comprising AAV of serotype 2 (AAV2), AAV of serotype 5 (AAV5), AAV of serotype 8 (AAV8), or AAV of serotype 9 (AAV9);
(b) a Rep/Cap nucleic acid molecule comprising a plasmid (pPackaging or pRC or pAAVRep/Cap) providing AAV viral functions and comprising a replication (Rep) gene from AAV2 and a capsid (Cap) gene from an AAV; and
(c) the chimeric helper nucleic acid molecule according to claim 1;
wherein the nucleic acid molecule (a), (b) and (c) are provided as one molecule, two different molecules or three different molecules.

12. A recombinant adeno-associated virus (rAAV) producer cell transformed with the packaging system according to claim 11.

13. The rAAV producer cell according to claim 12, which is a HEK-293 cell.

14. A helper-free virus method of producing an infectious recombinant adeno-associated virus (rAAV) in a producing cell line, comprising the steps of:
transforming, cells or a cell line with the packaging system according to claim 11 and allowing transfected cells to produce rAAV virions;
harvesting the transformed cells and lysing them to recover rAAV virions; and
collecting rAAV virions in cell lysates or supernatants and optionally purifying the rAAV virions.

15. The method according to claim 14, wherein the producing cell line is selected from the group consisting of HEK-293 cell line and wherein the cells are grown in suspension.

16. The method according to claim 14, wherein the transforming is transfecting and the transfection is carried out by chemical transfection, electroporation or sonoporation.

17. A method of producing a replication defective infectious recombinant adeno-associated virus (rAAV) vector particle, comprising the steps of:
transforming a cell with the packaging system according to claim 11 and allowing transformed cells to produce rAAV vector particles;
harvesting the transformed cells and lysing them to recover rAAV vector particles; and
collecting rAAV vector particles in cell lysates or supernatants and optionally purifying the rAAV vector particles.

18. In vitro use of the rAAV producer cell according to claim 12, in the production of a replication defective infectious rAAV vector particle.


SEQUENCE LISTING (36 sequences)

SEQ ID NO: 1 - DNA, 1878 bp, synthetic construct
  source 1..1878: mol_type = other DNA
  misc_feature 1..1878: note = Nucleotide sequence of the polynucleotide encoding the UL12 protein from HSV1
atggaaagcacggtaggtcccgcatgtcccccgggtagaacagtcaccaagcggccctgggccttggctgaggatacccctcgtggccctgatagcccgccaaaacgtcctaggcctaatagtcttccattgacgacaacattccgaccccttccaccacccccgcagaccacaagtgctgttgatccttccagtcactcccctgtcaatcccccacgcgatcaacatgccactgataccgccgatgagaaaccaagggcggctagcccggcattatccgatgcttcagggccgcccacccctgacatcccattgagcccaggcggtacacatgcaagggatccagacgctgacccagattcacccgacctcgactccatgtggtctgcttctgtgatccccaacgctctcccaagccacattttggccgaaacattcgaacgacatttaaggggcttacttaggggcgttcgcgcacctcttgcaataggacctttatgggctaggctcgattatctgtgttcactggccgtggtcctcgaggaagcagggatggtcgaccgtggtttagggcgccacctctggcgtttaaccagacgagggccaccagctgcagcagacgctgttgctccccggccattgatgggtttctacgaggctgccactcaaaaccaagccgattgtcaattgtgggccctgttgagaaggggcctgacaacagcaagcacactgagatggggccctcaaggaccgtgcttttcccctcagtggctcaaacacaacgcaagtctcaggcccgacgtccagagttctgcagttatgttcggacgggttaatgaacctacagcacgtagtctccttttcaggtactgcgtgggcagggctgatgatggtggtgaagcaggggccgacacgagaaggtttatctttcacgaaccttctgatctggccgaggagaatgttcacacatgcggcgtccttatggatggacacaccgggatggtcggggcttctctcgacatcctggtatgtccccgcgatatccatggctacctggcccccgtaccaaagacaccattggccttttatgaggtcaaatgtagagctaaatacgctttcgaccctatggatccgagcgatccaacagctagtgcatatgaggatttaatggcgcaccgctccccagaagcgtttcgtgcatttattcggtccattcctaagcccagcgttaggtattttgctcccggcagagttccaggccccgaagaagccctggtcacccaggatcaggcttggtccgaagcccatgccagcggggagaaacgtaggtgttctgctgccgacagggccttggtcgaactcaattctggcgtcgtgagcgaagttctgcttttcggagctcctgatttgggccgtcacactatttcccctgtgagctggtctagcggtgaccttgtacggagagagcccgttttcgcgaatcctcggcaccccaactttaaacaaatcctggttcagggctacgtgctggactcacattttcctgactgcccaccgcatccccacctggtgacctttataggacgacatagaacttcagccgaagaaggggtcaccttccgcctggaagatggcgctggagcactcggcgccgctggaccttcaaaggcttccattctccccaatcaagcggtcccgatagctttgatcatcaccccggtccgaatcgatccagaaatctacaaagcgatccaacggagcagcagattggccttcgacgatactctggctgaattatgggctagcaggagcccgggacctggccctgcagccgctgaaactacgagctcttcacccactactggaagatcttccaga

SEQ ID NO: 2 - DNA, 1419 bp, synthetic construct
  source 1..1419: mol_type = other DNA
  misc_feature 1..1419: note = Nucleotide sequence of the polynucleotide encoding the NS2 protein from HBoV1
atggccttcaacccccccgtgatcagagccttcagccagcccgccttcacctacgtgttcaagttcccctacccccagtggaaggagaaggagtggctgctgcacgccctgctggcccacggcaccgagcagagcatgatccagctgagaaactgcgccccccaccccgacgaggacatcatcagggacgacctgctgatcagcctggaggacagacacttcggcgccgtgctgtgcaaggccgtgtacatggccaccaccaccctgatgagccacaagcagagaaacatgttccccagatgcgacatcatcgtgcagagcgagctgggcgagaagaacctgcactgccacatcatcgtgggcggcgagggcctgagcaagagaaacgccaagagcagctgcgcccagttctacggcctgatcctggccgagatcatccagagatgcaagagcctgctggccaccagacccttcgagcccgaggaggccgacatcttccacaccctgaagaaggccgagagagaggcctggggcggcgtgaccggcggcaacatgcagatcctgcagtacagagacagaagaggcgacctgcacgcccagaccgtggaccccctgagattcttcaagaactacctgctgcccaagaacagatgcatcagcagctacagcaagcccgacgtgtgcaccagccccgacaactggttcatcctggccgagaagacctacagccacaccctgatcaacggcctgcccctgcccgagcactacagaaagaactaccacgccaccctggacaacgaggtgatccccggcccccagaccatggcctacggcggcagaggcccctgggagcacctgcccgaggacttcaccctgcacgagaacggctactgcaccgactgcggcggctacctgccccacagcgccgacaacagcatgtacaccgacagagccagcgagaccagcaccggcgacatcacccccagcgacctgggcgacagcgacggcgaggacaccgagcccgagaccagccaggtggactactgcccccccaagaagagaagactgaccgcccccgccagcccccccaacagccccgccagcagcgtgagcaccatcaccttcttcaacacctggcacgcccagcccagggacgaggacgagctgagagagtacgagagacaggccagcctgctgcagaagaagagagagagcagaaagagaggcgaggaggagaccctggccgacaacagcagccaggagcaggagccccagcccgaccccacccagtggggcgagagactgggcttcatcagcagcggcacccccaaccagccccccatcgtgctgcactgcttcgaggacctgagacccagcgacgaggacgagggcgagtacatcggcgagaagagacagtaa

SEQ ID NO: 3 - DNA, 487 bp, synthetic construct
  source 1..487: mol_type = other DNA
  misc_feature 1..487: note = Nucleotide sequence of VA-RNA from Ad5
ggtcgggacgctctggccggtcaggcgcgcgcaatcgttgacgctctagcgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagcccct

SEQ ID NO: 4 - DNA, 4734 bp, synthetic construct
  source 1..4734: mol_type = other DNA
  misc_feature 1..4734: note = Nucleotide sequence of the E2A gene from Ad5
tgacgtagttttcgcgcttaaatttgagaaagggcgcgaaactagtccttaagagtcagcgcgcagtatttactgaagagagcctccgcgtcttccagcgtgcgccgaagctgatcttcgcttttgtgatacaggcagctgcgggtgagggatcgcagagacctgttttttattttcagctcttgttcttggcccctgctctgttgaaatatagcatacagagtgggaaaaatcctgtttctaagctcgcgggtcgatacgggttcgttgggcgccagacgcagcgctcctcctcctgctgctgccgccgctgtggatttcttgggctttgtcagagtcttgctatccggtcgcctttgcttctgtgtggccgctgctgttgctgccgctgccgccggtgcagtatgggctgtagagatgacggtagtaatgcaggatgttacgggggaaggccacgccgtgatggtagagaagaaagcggcgggcgaaggagatgttgcccccacagtcttgcaagcaagcaactatggcgttcttgtgcccgcgccatgagcggtagccttggcgctgttgttgctcttgggctaacggcggcggctgcttggacttaccggccctggttccagtggtgtcccatctacggttgggtcggcgaacgggcagtgccggcggcgcctgaggagcggaggttgtagccatgctggaaccggttgccgatttctggggcgccggcgaggggaatgcgaccgagggtgacggtgtttcgtctgacacctcttcgacctcggaagcttcctcgtctaggctctcccagtcttccatcatgtcctcctcctcctcgtccaaaacctcctctgcctgactgtcccagtattcctcctcgtccgtgggtggcggcggcagctgcagcttctttttgggtgccatcctgggaagcaagggcccgcggctgctgctgatagggctgcggcggcggggggattgggttgagctcctcgccggactgggggtccaagtaaaccccccgtccctttcgtagcagaaactcttggcgggctttgttgatggcttgcaattggccaagaatgtggccctgggtaatgacgcaggcggtaagctccgcattaggcgggcgggattggtcttcgtagaacctaatctcgtgggcgtggtagtcctcaggtacaaatttgcgaaggtaagccgacgtccacagccccggagtgagtttcaaccccggagccgcggacttttcgtcaggcgagggaccctgcagctcaaaggtaccgataatttgactttcgttaagcagctgcgaattgcaaaccagggagcggtgcggggtgcataggttgcagcgacagtgacactccagtagaccgtcaccgctcacgtcttccattatgtcagagtggtaggcaaggtagttggctagctgcagaaggtagcagtggccccaaagcggcggagggcattcgcggtacttaatgggcacaaagtcgctaggaagtgcacagcaggtggcgggcaagattcctgagcgctctaggataaagttcctaaagttctgcaacatgctttgactggtgaagtctggcagaccctgttgcagggttttaagcaggcgttcggggaaaatgatgtccgccaggtgcgcggccacggagcgctcgttgaaggccgtccataggtccttcaagttttgctttagcagtttctgcagctccttgaggttgcactcctccaagcactgctgccaaacgcccatggccgtctgccaggtgtagcatagaaataagtaaacgcagtcgcggacgtagtcgcggcgcgcctcgcccttgagcgtggaatgaagcacgttttgcccaaggcggttttcgtgcaaaattccaaggtaggagaccaggttgcagagctccacgttggagatcttgcaggcctggcgtacgtagccctgtcgaaaggtgtagtgcaatgtttcctctagcttgcgctgcatctccgggtcagcaaagaaccgctgcatgcactcaagctccacggtaacgagcactgcggccatcattagtttgcgtcgctcctccaagtcggcaggctcgcgcgtttgaagccagcgcgctagctgctcgtcgccaactgcgggtaggccctcctctgtttgttcttgcaaatttgcatccctctccaggggctgcgcacggcgcacgatcagctcactcatgactgtgctcatgaccttggggggtaggttaagtgccgggtaggcaaagtgggtgacctcgatgctgcgttttagtacggctaggcgcgcgttgtcaccctcgagttccaccaacactccagagtgactttcattttcgctgttttcctgttgcagagcgtttgccgcgcgcttctcgtcgcgtccaagaccctcaaagatttttggcacttcgttgagcgaggcgatatcaggtatgacagcgccctgccgcaaggccagctgcttgtccgctcggctgcggttggcacggcaggataggggtatcttgcagttttggaaaaagatgtgataggtggcaagcacctctggcacggcaaatacggggtagaagttgaggcgcgggttgggctcgcatgtgccgttttcttggcgtttggggggtacgcgcggtgagaataggtggcgttcgtaggcaaggctgacatccgctatggcgaggggcacatcgctgcgctcttgcaacgcgtcgcagataatggcgcactggcgctgcagatgcttcaacagcacgtcatctcccacatctaggtagtcgccatgcctttcgtccccccgcccgacttgttcctcgtttgcctctgcgttgtcctggtcttgctttttatcctctgttggtactgagcggtcctcgtcgtcttcgcttacaaaacctgggtcctgctcgataatcacttcctcctcctcaagcgggggtgcctcgacggggaaggtggtaggcgcgttggcggcatcggtggaggcggtggtggcgaactcagagggggcggttaggctgtccttcttctcgactgactccatgatctttttctgcctataggagaaggaaatggccagtcgggaagaggagcagcgcgaaaccacccccgagcgcggacgcggtgcggcgcgacgtcccccaaccatggaggacgtgtcgtccccgtccccgtcgccgccgcctccccgggcgcccccaaaaaagcggatgaggcggcgtatcgagtccgaggacgaggaagactcatcacaagacgcgctggtgccgcgcacacccagcccgcggccatcgacctcggcggcggatttggccattgcgcccaagaagaaaaagaagcgcccttctcccaagcccgagcgcccgccatcaccagaggtaatcgtggacagcgaggaagaaagagaagatgtggcgctacaaatggtgggtttcagcaacccaccggtgctaatcaagcatggcaaaggaggtaagcgcacagtgcggcggctgaatgaagacgacccagtggcgcgtggtatgcggacgcaagaggaagaggaagagcccagcgaagcggaaagtgaaattacggtgatgaacccgctgagtgtgccgatcgtgtctgcgtgggagaagggcatggaggctgcgcgcgcgctgatggacaagtaccacgtggataacgatctaaaggcgaacttcaaactactgcctgaccaagtggaagctctggcggccgtatgcaagacctggctgaacgaggagcaccgcgggttgcagctgaccttcaccagcaacaagacctttgtgacgatgatggggcgattcctgcaggcgtacctgcagtcgtttgcagaggtgacctacaagcatcacgagcccacgggctgcgcgttgtggctgcaccgctgcgctgagatcgaaggcgagcttaagtgtctacacggaagcattatgataaataaggagcacgtgattgaaatggatgtgacgagcgaaaacgggcagcgcgcgctgaaggagcagtctagcaaggccaagatcgtgaagaaccggtggggccgaaatgtggtgcagatctccaacaccgacgcaaggtgctgcgtgcacgacgcggcctgtccggccaatcagttttccggcaagtcttgcggcatgttcttctctgaaggcgcaaaggctcaggtggcttttaagcagatcaaggcttttatgcaggcgctgtatcctaacgcccagaccgggcacggtcaccttttgatgccactacggtgcgagtgcaactcaaagcctgggcacgcgccctttttgggaaggcagctaccaaagttgactccgttcgccctgagcaacgcggaggacctggacgcggatctgatctccgacaagagcgtgctggccagcgtgcaccacccggcgctgatagtgttccagtgctgcaaccctgtgtatcgcaactcgcgcgcgcagggcggaggccccaactgcgacttcaagatatcggcgcccgacctgctaaacgcgttggtgatggtgcgcagcctgtggagtgaaaacttcaccgagctgccgcggatggttgtgcctgagtttaagtggagcactaaacaccagtatcgcaacgtgtccctgccagtggcgcatagcgatgcgcggcagaacccctttgatttttaaacggcgcagacggcaagggtgggggtaaataatcacccgagagtgtacaaataaa

SEQ ID NO: 5 - DNA, 3106 bp, synthetic construct
  source 1..3106: mol_type = other DNA
  misc_feature 1..3106: note = Nucleotide sequence of the E4 gene from Ad5
ttattttggattgaagccaatatgataatgagggggtggagtttgtgacgtggcgcggggcgtgggaacggggcgggtgacgtaggttttagggcggagtaacttgtatgtgttgggaattgtagttttcttaaaatgggaagtgacgtaacgtgggaaaacggaagtgacgatttgaggaagttgtgggttttttggctttcgtttctgggcgtaggttcgcgtgcggttttctgggtgttttttgtggactttaaccgttacgtcattttttagtcctatatatactcgctctgcacttggcccttttttacactgtgactgattgagctggtgccgtgtcgagtggtgtttttttaataggttttcttttttactggtaaggctgactgttatggctgccgctgtggaagcgctgtatgttgttctggagcgggagggtgctattttgcctaggcaggagggtttttcaggtgtttatgtgtttttctctcctattaattttgttatacctcctatgggggctgtaatgttgtctctacgcctgcgggtatgtattcccccgggctatttcggtcgctttttagcactgaccgatgtgaatcaacctgatgtgtttaccgagtcttacattatgactccggacatgaccgaggagctgtcggtggtgctttttaatcacggtgaccagtttttttacggtcacgccggcatggccgtagtccgtcttatgcttataagggttgtttttcctgttgtaagacaggcttctaatgtttaaatgtttttttgttattttattttgtgtttatgcagaaacccgcagacatgtttgagagaaaaatggtgtctttttctgtggtggttccggagcttacctgcctttatctgcatgagcatgactacgatgtgctttcttttttgcgcgaggctttgcctgattttttgagcagcaccttgcattttatatcgccgcccatgcaacaagcttacatcggggctacgctggttagcatagctccgagtatgcgtgtcataatcagtgtgggttcttttgtcatggttcctggcggggaagtggccgcgctggtccgtgcagacctgcacgattatgttcagctggccctgcgaagggacctacgggatcgcggtatttttgttaatgttccgcttttgaatcttatacaggtctgtgaggaacctgaatttttgcaatcatgattcgctgcttgaggctgaaggtggagggcgctctggagcagatttttacaatggccggacttaatattcgggatttgcttagagatatattgagaaggtggcgagatgagaattatttgggcatggttgaaggtgctggaatgtttatagaggagattcaccctgaagggtttagcctttacgtccacttggacgtgagggccgtttgccttttggaagccattgtgcaacatcttacaaatgccattatctgttctttggctgtagagtttgaccacgccaccggaggggagcgcgttcacttaatagatcttcattttgaggttttggataatcttttggaataaaaaaaaaaaacatggttcttccagctcttcccgctcctcccgtgtgtgactcgcagaacgaatgtgtaggttggctgggtgtggcttattctgcggtggtggatgttatcagggcagcggcgcatgaaggagtttacatagaacccgaagccagggggcgcctggatgctttgagagagtggatatactacaactactacacagagcgatctaagcggcgagaccggagacgcagatctgtttgtcacgcccgcacctggttttgcttcaggaaatatgactacgtccggcgttccatttggcatgacactacgaccaacacgatctcggttgtctcggcgcactccgtacagtagggatcgtctacctccttttgagacagaaacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctaacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgtatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgccttatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtaggcgtggacttctccttcgccgcccgttaagcaaccgcaagttggacagcagcctgtggctcagcagctggacagcgacatgaacttaagtgagctgcccggggagtttattaatatcactgatgagcgtttggctcgacaggaaaccgtgtggaatataacacctaagaatatgtctgttacccatgatatgatgctttttaaggccagccggggagaaaggactgtgtactctgtgtgttgggagggaggtggcaggttgaatactagggttctgtgagtttgattaaggtacggtgatctgtataagctatgtggtggtggggctatactactgaatgaaaaatgacttgaaattttctgcaattgaaaaataaa

SEQ ID NO: 6 - DNA, 1656 bp, synthetic construct
  source 1..1656: mol_type = other DNA
  misc_feature 1..1656: note = Nucleotide sequence of the E4orf6 sequence from Ad5
ttattttggattgaagccaatatgataatgagggggtggagtttgtgacgtggcgcggggcgtgggaacggggcgggtgacgtaggttttagggcggagtaacttgtatgtgttgggaattgtagttttcttaaaatgggaagtgacgtaacgtgggaaaacggaagtgacgatttgaggaagttgtgggttttttggctttcgtttctgggcgtaggttcgcgtgcggttttctgggtgttttttgtggactttaaccgttacgtcattttttagtcctatatatactcgctctgcacttggcccttttttacactgtgactgattgagctggtgccgtgtcgagtggtgtttttttaataggttttcttttttactggtaaggctgactgttatgactacgtccggcgttccatttggcatgacactacgaccaacacgatctcggttgtctcggcgcactccgtacagtagggatcgtctacctccttttgagacagaaacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctaacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgtatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgccttatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtaggcgtggacttctccttcgccgcccgttaagcaaccgcaagttggacagcagcctgtggctcagcagctggacagcgacatgaacttaagtgagctgcccggggagtttattaatatcactgatgagcgtttggctcgacaggaaaccgtgtggaatataacacctaagaatatgtctgttacccatgatatgatgctttttaaggccagccggggagaaaggactgtgtactctgtgtgttgggagggaggtggcaggttgaatactagggttctgtgagtttgattaaggtacggtgatctgtataagctatgtggtggtggggctatactactgaatgaaaaatgacttgaaattttctgcaattgaaaaataaa

SEQ ID NO: 7 - DNA, 894 bp, synthetic construct
  source 1..894: mol_type = other DNA
  misc_feature 1..894: note = Nucleotide sequence of insulated origin of replication
ccaggcatcaaataaaacgaaaggctcagtcgaaagactgggcctttcgttttatctgttgtttgtcggtgaacgctctctactagagtcacactggctcaccttcgggtgggcctttctgcgtttatatagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccagagaatataaaaagccagattattaatccggcttttttattattt

SEQ ID NO: 8 - DNA, 270 bp, synthetic construct
  source 1..270: mol_type = other DNA
  misc_feature 1..270: note = Nucleotide sequence of HSV TK polyA signal
gggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgaggccccattggggccaatacgcccgcgtttcttccttttccccaccccaccccccaagttcgggtgaaggcccagggctcgcagccaacgtcggggcggcaggccctgccatag

SEQ ID NO: 9 - DNA, 228 bp, synthetic construct
  source 1..228: mol_type = other DNA
  misc_feature 1..228: note = Nucleotide sequence of bGH polyA signal
cgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaggacaatagcaggcatgctggggatgcggtgggctctatgg

SEQ ID NO: 10 - DNA, 527 bp, synthetic construct
  source 1..527: mol_type = other DNA
  misc_feature 1..527: note = Nucleotide sequence of the CMV promoter
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcaga

SEQ ID NO: 11 - DNA, 304 bp, synthetic construct
  source 1..304: mol_type = other DNA
  misc_feature 1..304: note = Nucleotide sequence of the CMV enhancer
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 12 - DNA, 231 bp, synthetic construct
  source 1..231: mol_type = other DNA
  misc_feature 1..231: note = Nucleotide sequence of the EFS promoter
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 13 - DNA, 342 bp, synthetic construct
  source 1..342: mol_type = other DNA
  misc_feature 1..342: note = Nucleotide sequence of the E2A promoter
tgacgtagttttcgcgcttaaatttgagaaagggcgcgaaactagtccttaagagtcagcgcgcagtatttactgaagagagcctccgcgtcttccagcgtgcgccgaagctgatcttcgcttttgtgatacaggcagctgcgggtgagggatcgcagagacctgttttttattttcagctcttgttcttggcccctgctctgttgaaatatagcatacagagtgggaaaaatcctgtttctaagctcgcgggtcgatacgggttcgttgggcgccagacgcagcgctcctcctcctgctgctgccgccgctgtggatttcttgggctttgtcagagtcttg

SEQ ID NO: 14 - DNA, 394 bp, synthetic construct
  source 1..394: mol_type = other DNA
  misc_feature 1..394: note = Nucleotide sequence of the E4 promoter
ttattttggattgaagccaatatgataatgagggggtggagtttgtgacgtggcgcggggcgtgggaacggggcgggtgacgtaggttttagggcggagtaacttgtatgtgttgggaattgtagttttcttaaaatgggaagtgacgtaacgtgggaaaacggaagtgacgatttgaggaagttgtgggttttttggctttcgtttctgggcgtaggttcgcgtgcggttttctgggtgttttttgtggactttaaccgttacgtcattttttagtcctatatatactcgctctgcacttggcccttttttacactgtgactgattgagctggtgccgtgtcgagtggtgtttttttaataggttttcttttttactggtaaggctgactgtt

SEQ ID NO: 15 - DNA, 952 bp, synthetic construct
  source 1..952: mol_type = other DNA
  misc_feature 1..952: note = Nucleotide sequence of the kanamycin resistance gene
cgcggaacccctatttgtttatttttctaaatacattcaaatatgtatccgctcatgagacaataaccctgataaatgcttcaataatattgaaaaaggaagagtgaaggagaatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaataaccccttggggcctctaaacgggtcttgaggggttttttga

SEQ ID NO: 16 - DNA, 2392 bp, synthetic construct
  source 1..2392: mol_type = other DNA
  misc_feature 1..2392: note = Polynucleotide insert comprising the EFS promoter, the polynucleotide encoding the UL12 protein from HSV1 and the HSV TK polyA signal
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggccaccatggaaagcacggtaggtcccgcatgtcccccgggtagaacagtcaccaagcggccctgggccttggctgaggatacccctcgtggccctgatagcccgccaaaacgtcctaggcctaatagtcttccattgacgacaacattccgaccccttccaccacccccgcagaccacaagtgctgttgatccttccagtcactcccctgtcaatcccccacgcgatcaacatgccactgataccgccgatgagaaaccaagggcggctagcccggcattatccgatgcttcagggccgcccacccctgacatcccattgagcccaggcggtacacatgcaagggatccagacgctgacccagattcacccgacctcgactccatgtggtctgcttctgtgatccccaacgctctcccaagccacattttggccgaaacattcgaacgacatttaaggggcttacttaggggcgttcgcgcacctcttgcaataggacctttatgggctaggctcgattatctgtgttcactggccgtggtcctcgaggaagcagggatggtcgaccgtggtttagggcgccacctctggcgtttaaccagacgagggccaccagctgcagcagacgctgttgctccccggccattgatgggtttctacgaggctgccactcaaaaccaagccgattgtcaattgtgggccctgttgagaaggggcctgacaacagcaagcacactgagatggggccctcaaggaccgtgcttttcccctcagtggctcaaacacaacgcaagtctcaggcccgacgtccagagttctgcagttatgttcggacgggttaatgaacctacagcacgtagtctccttttcaggtactgcgtgggcagggctgatgatggtggtgaagcaggggccgacacgagaaggtttatctttcacgaaccttctgatctggccgaggagaatgttcacacatgcggcgtccttatggatggacacaccgggatggtcggggcttctctcgacatcctggtatgtccccgcgatatccatggctacctggcccccgtaccaaagacaccattggccttttatgaggtcaaatgtagagctaaatacgctttcgaccctatggatccgagcgatccaacagctagtgcatatgaggatttaatggcgcaccgctccccagaagcgtttcgtgcatttattcggtccattcctaagcccagcgttaggtattttgctcccggcagagttccaggccccgaagaagccctggtcacccaggatcaggcttggtccgaagcccatgccagcggggagaaacgtaggtgttctgctgccgacagggccttggtcgaactcaattctggcgtcgtgagcgaagttctgcttttcggagctcctgatttgggccgtcacactatttcccctgtgagctggtctagcggtgaccttgtacggagagagcccgttttcgcgaatcctcggcaccccaactttaaacaaatcctggttcagggctacgtgctggactcacattttcctgactgcccaccgcatccccacctggtgacctttataggacgacatagaacttcagccgaagaaggggtcaccttccgcctggaagatggcgctggagcactcggcgccgctggaccttcaaaggcttccattctccccaatcaagcggtcccgatagctttgatcatcaccccggtccgaatcgatccagaaatctacaaagcgatccaacggagcagcagattggccttcgacgatactctggctgaattatgggctagcaggagcccgggacctggccctgcagccgctgaaactacgagctcttcacccactactggaagatcttccagataaacttgggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgaggccccattggggccaatacgcccgcgtttcttccttttccccaccccaccccccaagttcgggtgaaggcccagggctcgcagccaacgtcggggcggcaggccctgccatag

SEQ ID NO: 17 - DNA, 2184 bp, synthetic construct
  source 1..2184: mol_type = other DNA
  misc_feature 1..2184: note = Polynucleotide insert comprising the CMV promoter, the polynucleotide encoding the NS2 protein from HBoV1 and the bGH polyA signal
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagagccaccatggccttcaacccccccgtgatcagagccttcagccagcccgccttcacctacgtgttcaagttcccctacccccagtggaaggagaaggagtggctgctgcacgccctgctggcccacggcaccgagcagagcatgatccagctgagaaactgcgccccccaccccgacgaggacatcatcagggacgacctgctgatcagcctggaggacagacacttcggcgccgtgctgtgcaaggccgtgtacatggccaccaccaccctgatgagccacaagcagagaaacatgttccccagatgcgacatcatcgtgcagagcgagctgggcgagaagaacctgcactgccacatcatcgtgggcggcgagggcctgagcaagagaaacgccaagagcagctgcgcccagttctacggcctgatcctggccgagatcatccagagatgcaagagcctgctggccaccagacccttcgagcccgaggaggccgacatcttccacaccctgaagaaggccgagagagaggcctggggcggcgtgaccggcggcaacatgcagatcctgcagtacagagacagaagaggcgacctgcacgcccagaccgtggaccccctgagattcttcaagaactacctgctgcccaagaacagatgcatcagcagctacagcaagcccgacgtgtgcaccagccccgacaactggttcatcctggccgagaagacctacagccacaccctgatcaacggcctgcccctgcccgagcactacagaaagaactaccacgccaccctggacaacgaggtgatccccggcccccagaccatggcctacggcggcagaggcccctgggagcacctgcccgaggacttcaccctgcacgagaacggctactgcaccgactgcggcggctacctgccccacagcgccgacaacagcatgtacaccgacagagccagcgagaccagcaccggcgacatcacccccagcgacctgggcgacagcgacggcgaggacaccgagcccgagaccagccaggtggactactgcccccccaagaagagaagactgaccgcccccgccagcccccccaacagccccgccagcagcgtgagcaccatcaccttcttcaacacctggcacgcccagcccagggacgaggacgagctgagagagtacgagagacaggccagcctgctgcagaagaagagagagagcagaaagagaggcgaggaggagaccctggccgacaacagcagccaggagcaggagccccagcccgaccccacccagtggggcgagagactgggcttcatcagcagcggcacccccaaccagccccccatcgtgctgcactgcttcgaggacctgagacccagcgacgaggacgagggcgagtacatcggcgagaagagacagtaacggtcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaggacaatagcaggcatgctggggatgcggtgggctctatgg

SEQ ID NO: 18 - DNA, 13324 bp, synthetic construct
  source 1..13324: mol_type = other DNA
  misc_feature 1..13324: note = Plasmid Ad-HBoV-HSV
tccaggcatcaaataaaacgaaaggctcagtcgaaagactgggcctttcgttttatctgttgtttgtcggtgaacgctctctactagagtcacactggctcaccttcgggtgggcctttctgcgtttatatagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccagagaatataaaaagccagattattaatccggcttttttattattttgacctatggcagggcctgccgccccgacgttggctgcgagccctgggccttcacccgaacttggggggtggggtggggaaaaggaagaaacgcgggcgtattggccccaatggggcctcggtggggtatcgacagagtgccagccctgggaccgaaccccgcgtttatgaacaaacgacccaacaccgtgcgttttattctgtctttttattgccgtcatagcgcgggttccttccggtattgtctccttccgtgtttcagttagcctcccccaagtttatctggaagatcttccagtagtgggtgaagagctcgtagtttcagcggctgcagggccaggtcccgggctcctgctagcccataattcagccagagtatcgtcgaaggccaatctgctgctccgttggatcgctttgtagatttctggatcgattcggaccggggtgatgatcaaagctatcgggaccgcttgattggggagaatggaagcctttgaaggtccagcggcgccgagtgctccagcgccatcttccaggcggaaggtgaccccttcttcggctgaagttctatgtcgtcctataaaggtcaccaggtggggatgcggtgggcagtcaggaaaatgtgagtccagcacgtagccctgaaccaggatttgtttaaagttggggtgccgaggattcgcgaaaacgggctctctccgtacaaggtcaccgctagaccagctcacaggggaaatagtgtgacggcccaaatcaggagctccgaaaagcagaacttcgctcacgacgccagaattgagttcgaccaaggccctgtcggcagcagaacacctacgtttctccccgctggcatgggcttcggaccaagcctgatcctgggtgaccagggcttcttcggggcctggaactctgccgggagcaaaatacctaacgctgggcttaggaatggaccgaataaatgcacgaaacgcttctggggagcggtgcgccattaaatcctcatatgcactagctgttggatcgctcggatccatagggtcgaaagcgtatttagctctacatttgacctcataaaaggccaatggtgtctttggtacgggggccaggtagccatggatatcgcggggacataccaggatgtcgagagaagccccgaccatcccggtgtgtccatccataaggacgccgcatgtgtgaacattctcctcggccagatcagaaggttcgtgaaagataaaccttctcgtgtcggcccctgcttcaccaccatcatcagccctgcccacgcagtacctgaaaaggagactacgtgctgtaggttcattaacccgtccgaacataactgcagaactctggacgtcgggcctgagacttgcgttgtgtttgagccactgaggggaaaagcacggtccttgagggccccatctcagtgtgcttgctgttgtcaggccccttctcaacagggcccacaattgacaatcggcttggttttgagtggcagcctcgtagaaacccatcaatggccggggagcaacagcgtctgctgcagctggtggccctcgtctggttaaacgccagaggtggcgccctaaaccacggtcgaccatccctgcttcctcgaggaccacggccagtgaacacagataatcgagcctagcccataaaggtcctattgcaagaggtgcgcgaacgcccctaagtaagccccttaaatgtcgttcgaatgtttcggccaaaatgtggcttgggagagcgttggggatcacagaagcagaccacatggagtcgaggtcgggtgaatctgggtcagcgtctggatcccttgcatgtgtaccgcctgggctcaatgggatgtcaggggtgggcggccctgaagcatcggataatgccgggctagccgcccttggtttctcatcggcggtatcagtggcatgttgatcgcgtgggggattgacaggggagtgactggaaggatcaacagcacttgtggtctgcgggggtggtggaaggggtcggaatgttgtcgtcaatggaagactattaggcctaggacgttttggcgggctatcagggccacgaggggtatcctcagccaaggcccagggccgcttggtgactgttctacccgggggacatgcgggacctaccgtgctttccatggtggcctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccactgacgggcaccggagccgcctccatagagcccaccgcatccccagcatgcctgctattgtcctcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaccgttactgtctcttctcgccgatgtactcgccctcgtcctcgtcgctgggtctcaggtcctcgaagcagtgcagcacgatggggggctggttgggggtgccgctgctgatgaagcccagtctctcgccccactgggtggggtcgggctggggctcctgctcctggctgctgttgtcggccagggtctcctcctcgcctctctttctgctctctctcttcttctgcagcaggctggcctgtctctcgtactctctcagctcgtcctcgtccctgggctgggcgtgccaggtgttgaagaaggtgatggtgctcacgctgctggcggggctgttgggggggctggcgggggcggtcagtcttctcttcttgggggggcagtagtccacctggctggtctcgggctcggtgtcctcgccgtcgctgtcgcccaggtcgctgggggtgatgtcgccggtgctggtctcgctggctctgtcggtgtacatgctgttgtcggcgctgtggggcaggtagccgccgcagtcggtgcagtagccgttctcgtgcagggtgaagtcctcgggcaggtgctcccaggggcctctgccgccgtaggccatggtctgggggccggggatcacctcgttgtccagggtggcgtggtagttctttctgtagtgctcgggcaggggcaggccgttgatcagggtgtggctgtaggtcttctcggccaggatgaaccagttgtcggggctggtgcacacgtcgggcttgctgtagctgctgatgcatctgttcttgggcagcaggtagttcttgaagaatctcagggggtccacggtctgggcgtgcaggtcgcctcttctgtctctgtactgcaggatctgcatgttgccgccggtcacgccgccccaggcctctctctcggccttcttcagggtgtggaagatgtcggcctcctcgggctcgaagggtctggtggccagcaggctcttgcatctctggatgatctcggccaggatcaggccgtagaactgggcgcagctgctcttggcgtttctcttgctcaggccctcgccgcccacgatgatgtggcagtgcaggttcttctcgcccagctcgctctgcacgatgatgtcgcatctggggaacatgtttctctgcttgtggctcatcagggtggtggtggccatgtacacggccttgcacagcacggcgccgaagtgtctgtcctccaggctgatcagcaggtcgtccctgatgatgtcctcgtcggggtggggggcgcagtttctcagctggatcatgctctgctcggtgccgtgggccagcagggcgtgcagcagccactccttctccttccactgggggtaggggaacttgaacacgtaggtgaaggcgggctggctgaaggctctgatcacgggggggttgaaggccatggtggctctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgttttggtcgggacgctctggccggtcaggcgcgcgcaatcgttgacgctctagcgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagcccctatcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagatgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcaaacgttattttggattgaagccaatatgataatgagggggtggagtttgtgacgtggcgcggggcgtgggaacggggcgggtgacgtaggttttagggcggagtaacttgtatgtgttgggaattgtagttttcttaaaatgggaagtgacgtaacgtgggaaaacggaagtgacgatttgaggaagttgtgggttttttggctttcgtttctgggcgtaggttcgcgtgcggttttctgggtgttttttgtggactttaaccgttacgtcattttttagtcctatatatactcgctctgcacttggcccttttttacactgtgactgattgagctggtgccgtgtcgagtggtgtttttttaataggttttcttttttactggtaaggctgactgttatgactacgtccggcgttccatttggcatgacactacgaccaacacgatctcggttgtctcggcgcactccgtacagtagggatcgtctacctccttttgagacagaaacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctaacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgtatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgccttatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtaggcgtggacttctccttcgccgcccgttaagcaaccgcaagttggacagcagcctgtggctcagcagctggacagcgacatgaacttaagtgagctgcccggggagtttattaatatcactgatgagcgtttggctcgacaggaaaccgtgtggaatataacacctaagaatatgtctgttacccatgatatgatgctttttaaggccagccggggagaaaggactgtgtactctgtgtgttgggagggaggtggcaggttgaatactagggttctgtgagtttgattaaggtacggtgatctgtataagctatgtggtggtggggctatactactgaatgaaaaatgacttgaaattttctgcaattgaaaaataaattttcgcggaacccctatttgtttatttttctaaatacattcaaatatgtatccgctcatgagacaataaccctgataaatgcttcaataatattgaaaaaggaagagtgaaggagaatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaataaccccttggggcctctaaacgggtcttgaggggttttttga

SEQ ID NO: 19 - protein, 626 aa, synthetic construct
  REGION 1..626: note = UL12 protein from HSV1
MESTVGPACPPGRTVTKRPWALAEDTPRGPDSPPKRPRPNSLPLTTTFRPLPPPPQTTSAVDPSSHSPVNPPRDQHATDTADEKPRAASPALSDASGPPTPDIPLSPGGTHARDPDADPDSPDLDSMWSASVIPNALPSHILAETFERHLRGLLRGVRAPLAIGPLWARLDYLCSLAVVLEEAGMVDRGLGRHLWRLTRRGPPAAADAVAPRPLMGFYEAATQNQADCQLWALLRRGLTTASTLRWGPQGPCFSPQWLKHNASLRPDVQSSAVMFGRVNEPTARSLLFRYCVGRADDGGEAGADTRRFIFHEPSDLAEENVHTCGVLMDGHTGMVGASLDILVCPRDIHGYLAPVPKTPLAFYEVKCRAKYAFDPMDPSDPTASAYEDLMAHRSPEAFRAFIRSIPKPSVRYFAPGRVPGPEEALVTQDQAWSEAHASGEKRRCSAADRALVELNSGVVSEVLLFGAPDLGRHTISPVSWSSGDLVRREPVFANPRHPNFKQILVQGYVLDSHFPDCPPHPHLVTFIGRHRTSAEEGVTFRLEDGAGALGAAGPSKASILPNQAVPIALIITPVRIDPEIYKAIQRSSRLAFDDTLAELWASRSPGPGPAAAETTSSSPTTGRSSR

SEQ ID NO: 20 - protein, 472 aa, synthetic construct
  REGION 1..472: note = NS2 protein from HBoV1
MAFNPPVIRAFSQPAFTYVFKFPYPQWKEKEWLLHALLAHGTEQSMIQLRNCAPHPDEDIIRDDLLISLEDRHFGAVLCKAVYMATTTLMSHKQRNMFPRCDIIVQSELGEKNLHCHIIVGGEGLSKRNAKSSCAQFYGLILAEIIQRCKSLLATRPFEPEEADIFHTLKKAEREAWGGVTGGNMQILQYRDRRGDLHAQTVDPLRFFKNYLLPKNRCISSYSKPDVCTSPDNWFILAEKTYSHTLINGLPLPEHYRKNYHATLDNEVIPGPQTMAYGGRGPWEHLPEDFTLHENGYCTDCGGYLPHSADNSMYTDRASETSTGDITPSDLGDSDGEDTEPETSQVDYCPPKKRRLTAPASPPNSPASSVSTITFFNTWHAQPRDEDELREYERQASLLQKKRESRKRGEEETLADNSSQEQEPQPDPTQWGERLGFISSGTPNQPPIVLHCFEDLRPSDEDEGEYIGEKRQ

SEQ ID NO: 21 - protein, 529 aa, synthetic construct
  REGION 1..529: note = E2A protein from Ad5
MASREEEQRETTPERGRGAARRPPTMEDVSSPSPSPPPPRAPPKKRMRRRIESEDEEDSSQDALVPRTPSPRPSTSAADLAIAPKKKKKRPSPKPERPPSPEVIVDSEEEREDVALQMVGFSNPPVLIKHGKGGKRTVRRLNEDDPVARGMRTQEEEEEPSEAESEITVMNPLSVPIVSAWEKGMEAARALMDKYHVDNDLKANFKLLPDQVEALAAVCKTWLNEEHRGLQLTFTSNKTFVTMMGRFLQAYLQSFAEVTYKHHEPTGCALWLHRCAEIEGELKCLHGSIMINKEHVIEMDVTSENGQRALKEQSSKAKIVKNRWGRNVVQISNTDARCCVHDAACPANQFSGKSCGMFFSEGAKAQVAFKQIKAFMQALYPNAQTGHGHLLMPLRCECNSKPGHAPFLGRQLPKLTPFALSNAEDLDADLISDKSVLASVHHPALIVFQCCNPVYRNSRAQGGGPNCDFKISAPDLLNALVMVRSLWSENFTELPRMVVPEFKWSTKHQYRNVSLPVAHSDARQNPFDF

SEQ ID NO: 22 - protein, 128 aa, synthetic construct
  REGION 1..128: note = E4orf1 sequence from Ad5
MAAAVEALYVVLEREGAILPRQEGFSGVYVFFSPINFVIPPMGAVMLSLRLRVCIPPGYFGRFLALTDVNQPDVFTESYIMTPDMTEELSVVLFNHGDQFFYGHAGMAVVRLMLIRVVFPVVRQASNV

SEQ ID NO: 23 - protein, 294 aa, synthetic construct
  REGION 1..294: note = E4orf6 sequence from Ad5
MTTSGVPFGMTLRPTRSRLSRRTPYSRDRLPPFETETRATILEDHPLLPECNTLTMHNVSYVRGLPCSVGFTLIQEWVVPWDMVLTREELVILRKCMHVCLCCANIDIMTSMMIHGYESWALHCHCSSPGSLQCIAGGQVLASWFRMVVDGAMFNQRFIWYREVVNYNMPKEVMFMSSVFMRGRHLIYLRLWYDGHVGSVVPAMSFGYSALHCGILNNIVVLCCSYCADLSEIRVRCCARRTRRLMLRAVRIIAEETTAMLYSCRTERRRQQFIRALLQHHRPILMHDYDSTPM

SEQ ID NO: 24 - DNA, 660 bp, synthetic construct
  source 1..660: mol_type = other DNA
  misc_feature 1..660: note = Nucleotide sequence of the polynucleotide encoding the NP1 protein from HBoV1
atgagctcagggaatatgaaagacaagcatcgctcctacaaaagaaaagggagtccagaaagaggggagaggaagagacactggcagacaactcatcacaggagcaggagccgcagcccgatccgacacagtggggagagaggctcgggctcatatcatcaggaacacccaatcagccacctatcgtcttgcactgcttcgaagacctcagaccaagtgatgaagacgagggagagtacatcggggaaaaaagacaatagaacaaatccatacactgtattcagtcaacacagagcttccaatcctgaagctccagggtggtgtgggttctactggcactctactcgcattgctagagatggtactaattcaatctttaatgaaatgaaacaacagtttcaacagctacaaattgataataaaataggatgggataacactagagaactattgtttaatcaaaagaaaacactagatcaaaaatacagaaatatgttctggcactttagaaataactctgattgtgaaagatgtaattactgggatgatgtgtaccgtagacacttagctaatgtttcctcacagacagaagcagacgagataactgacgaggaaatgctttctgctgctgaaagcatggaagcagatgcctccaattaa

SEQ ID NO: 25 - protein, 219 aa, synthetic construct
  REGION 1..219: note = NP1 protein from HBoV1
MSSGNMKDKHRSYKRKGSPERGERKRHWQTTHHRSRSRSPIRHSGERGSGSYHQEHPISHLSSCTASKTSDQVMKTRESTSGKKDNRTNPYTVFSQHRASNPEAPGWCGFYWHSTRIARDGTNSIFNEMKQQFQQLQIDNKIGWDNTRELLFNQKKTLDQKYRNMFWHFRNNSDCERCNYWDDVYRRHLANVSSQTEADEITDEEMLSAAESMEADASN

SEQ ID NO: 26 - DNA, 3408 bp, synthetic construct
  source 1..3408: mol_type = other DNA
  misc_feature 1..3408: note = Nucleotide sequence of the E1 gene from Ad5
gaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagctgacgtgtagtgtatttatacccggtgagttcctcaagaggccactcttgagtgccagcgagtagagttttctcctccgagccgctccgacaccgggactgaaaatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataaggtgtaaacctgtgattgcgtgtgtggttaacgcctttgtttgctgaatgagttgatgtaagtttaataaagggtgagataatgtttaacttgcatggcgtgttaaatggggcggggcttaaagggtatataatgcgccgtgggctaatcttggttacatctgacctcatggaggcttgggagtgtttggaagatttttctgctgtgcgtaacttgctggaacagagctctaacagtacctcttggttttggaggtttctgtggggctcatcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgaatgttgtacaggtggctgaactgtatccagaactgagacgcattttgacaattacagaggatgggcaggggctaaagggggtaaagagggagcggggggcttgtgaggctacagaggaggctaggaatctagcttttagcttaatgaccagacaccgtcctgagtgtattacttttcaacagatcaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagatcagcaaacttgtaaatatcaggaattgttgctacatttctgggaacggggccgaggtggagatagatacggaggatagggtggcctttagatgtagcatgataaatatgtggccgggggtgcttggcatggacggggtggttattatgaatgtaaggtttactggccccaattttagcggtacggttttcctggccaataccaaccttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctctttgaaaggtgtaccttgggtatcctgtctgagggtaactccagggtgcgccacaatgtggcctccgactgtggttgcttcatgctagtgaaaagcgtggctgtgattaagcataacatggtatgtggcaactgcgaggacagggcctctcagatgctgacctgctcggacggcaactgtcacctgctgaagaccattcacgtagccagccactctcgcaaggcctggccagtgtttgagcataacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatgtgtgggcgtggcttaagggtgggaaagaatatataaggtgggggtcttatgtagttttgtatctgttttgcagcagccgccgccgcc

SEQ ID NO: 27 - protein, 186 aa, synthetic construct
  REGION 1..186: note = E1A protein from Ad5
MRHIICHGGVITEEMAASLLDQLIEEVLADNLPPPSHFEPPTLHELYDLDVTAPEDPNEEAVSQIFPDSVMLAVQEGIDLLTFPPAPGSPEPPHLSRQPEQPEQRALGPVSMPNLVPEVIDLTCHEAGFPPSDDEDEEGEEFVLDYVEHPGHGCRSCHYHRRNTGDPDIMCSLCYMRTCGMFVYSK

SEQ ID NO: 28 - protein, 496 aa, synthetic construct
  REGION 1..496: note = E1B55k protein from Ad5
MERRNPSERGVPAGFSGHASVESGCETQESPATVVFRPPGDNTDGGAAAAAGGSQAAAAGAEPMEPESRPGPSGMNVVQVAELYPELRRILTITEDGQGLKGVKRERGACEATEEARNLAFSLMTRHRPECITFQQIKDNCANELDLLAQKYSIEQLTTYWLQPGDDFEEAIRVYAKVALRPDCKYKISKLVNIRNCCYISGNGAEVEIDTEDRVAFRCSMINMWPGVLGMDGVVIMNVRFTGPNFSGTVFLANTNLILHGVSFYGFNNTCVEAWTDVRVRGCAFYCCWKGVVCRPKSRASIKKCLFERCTLGILSEGNSRVRHNVASDCGCFMLVKSVAVIKHNMVCGNCEDRASQMLTCSDGNCHLLKTIHVASHSRKAWPVFEHNILTRCSLHLGNRRGVFLPYQCNLSHTKILLEPESMSKVNLNGVFDMTMKIWKVLRYDETRTRCRPCECGGKHIRNQPVMLDVTEELRPDHLVLACTRAEFGSSDEDTD

SEQ ID NO: 29 - protein, 136 aa, synthetic construct
  REGION 1..136: note = E4orf2 sequence from Ad5
MQKPADMFERKMVSFSVVVPELTCLYLHEHDYDVLSFLREALPDFLSSTLHFISPPMQQAYIGATLVSIAPSMRVIISVGSFVMVPGGEVAALVRADLHDYVQLALRRDLRDRGIFVNVPLLNLIQVCEEPEFLQS

SEQ ID NO: 30 - protein, 116 aa, synthetic construct
  REGION 1..116: note = E4orf3 sequence from Ad5
MIRCLRLKVEGALEQIFTMAGLNIRDLLRDILRRWRDENYLGMVEGAGMFIEEIHPEGFSLYVHLDVRAVCLLEAIVQHLTNAIICSLAVEFDHATGGERVHLIDLHFEVLDNLLE

SEQ ID NO: 31 - protein, 114 aa, synthetic construct
  REGION 1..114: note = E4orf4 sequence from Ad5
MVLPALPAPPVCDSQNECVGWLGVAYSAVVDVIRAAAHEGVYIEPEARGRLDALREWIYYNYYTERSKRRDRRRRSVCHARTWFCFRKYDYVRRSIWHDTTTNTISVVSAHSVQ

SEQ ID NO: 32 - DNA, 3588 bp, synthetic construct
  source 1..3588: mol_type = other DNA
  misc_feature 1..3588: note = Nucleotide sequence of the polynucleotide encoding the ICP8 protein from HSV1
atggaaaccaagcccaaaacagcaacgactatcaaggtgccccctggacccctggggtacgtatatgctcgggcctgtcctagcgaggggatagaattgttggccttgttgagtgcccgaagcggggactctgatgtcgcagtggccccactcgtggtggggcttaccgtagaaagtgggttcgaggctaatgttgctgtggtcgtgggctcaagaaccacggggcttggcggaactgcagtttcccttaaactgacacccagtcactattcatcttctgtatacgtcttccacggtgggcgccaccttgatccctcaacgcaggcacctaatctcaccaggctctgcgaaagggcccggcgacattttggctttagtgactatacgcccagacctggtgatctgaagcatgaaactacgggagaggcactttgtgaaagacttgggctggatcccgatcgtgcgttgctgtacctcgtggtaaccgaaggttttaaagaggccgtctgcatcaataatacttttctgcatttgggcggtagcgacaaagtgacaatcggtggcgctgaggttcaccgaatccccgtctacccgcttcagctcttcatgccggacttttccagggtgatcgccgagccattcaatgccaatcacaggtccataggagaaaagtttacctaccctctccccttctttaacaggccccttaaccggttgctgtttgaagctgtggtcggcccagctgcggtagcactgaggtgtaggaatgtcgatgcggtagcaagagctgcagcccatttggcctttgatgaaaaccacgaaggtgcggcactgcccgcagacataacatttaccgcctttgaggcgagtcaaggcaaaactccccgcggcggacgggacggtggtggtaaaggcgcagccggcggctttgagcagagattagcctctgtcatggccggagatgcggcactggcactggagagcatagtatccatggccgttttcgatgaacccccaaccgacatttccgcctggccactgtttgaagggcaggatactgctgcagcgcgcgccaacgccgttggcgcatatttggcccgggcagctggactggtcggagcgatggtgttcagcaccaatagcgcccttcatctgaccgaggtcgatgacgcgggcccagccgaccctaaagatcattccaaacccagcttttacaggttcttcctggtaccaggcactcatgtggcagcgaatcctcaagttgaccgggaaggacatgtggtccctggctttgagggaagacccacagctccgctggtgggcggcactcaagaatttgcgggagagcaccttgcaatgctttgcggcttctctccggcactccttgctaagatgttgttctatctcgagaggtgtgacggggccgtgatagtcggcagacaggagatggacgtcttccgatatgtggcagacagcaatcaaactgatgtgccttgcaatctctgtacattcgacacacgccatgcttgcgtccacacaactctcatgcgcctgcgcgctagacatcctaaatttgcgagtgctgccaggggtgccattggtgtattcggaacgatgaactcaatgtattccgattgcgatgtactggggaattacgccgcattttcagctcttaaaagagctgacggatcagaaacagcaagaacgattatgcaagaaacgtatcgcgctgcaactgaacgcgttatggccgagttggaaacgctccaatatgttgatcaagcagtgcccaccgccatgggacgccttgagacaattattaccaacagggaagccttgcacaccgtcgtaaacaacgtccggcaagtggtggatcgcgaagtcgaacaacttatgagaaacctcgttgaaggcaggaatttcaaatttagggacggtctgggcgaagctaaccatgctatgtctctgacgctcgatccatatgcgtgtgggccatgtccactgctgcaacttctggggcgcaggagcaatttagcggtgtaccaggatctcgccctttcccaatgtcatggagttttcgccggacaatccgtcgaaggtcgcaatttcaggaaccaatttcaaccagtactgagaaggcgagtgatggatatgttcaataatgggttcctctccgccaaaactctcacagtggctctgtcagagggcgcagccatatgcgcacccagccttactgctgggcaaaccgccccagctgaatcaagcttcgaaggtgatgtcgcgcgagtcaccctcggctttcccaaggaacttagagtcaagtccagggtcctgtttgctggcgcttctgcaaatgcctctgaagccgccaaggctagagttgcaagtctgcagagcgcctaccaaaagcctgataagagagtcgatatcctgcttgggccgttgggattcttgctcaaacaattccacgcagcaattttccccaatgggaaaccacctggctccaatcaacctaatccgcaatggttttggaccgccttgcaacgcaatcagctgccagcacggctcctgtcccgcgaggatattgagactatcgcgttcatcaagaaattttccctcgactacggcgccataaacttcattaacttggctcctaacaatgtttctgaattggctatgtactatatggctaaccaaatcctgaggtactgtgatcactcaacatacttcataaatacgcttaccgctatcatagcgggtagccgccggcccccttcagttcaagcagcagccgcttggtcagcgcaaggtggtgctgggctggaagcaggcgcacgtgctctgatggacgcagtggatgcacatcctggcgcttggacaagtatgtttgcgagttgtaacttgcttagacctgtaatggcggcaagaccaatggtcgttttaggcttgagcatatctaagtattacggcatggcaggaaatgatcgtgtgtttcaagccggcaactgggcttctctgatgggcggtaagaacgcctgcccgctgctgattttcgaccggactcgaaaatttgtacttgcctgtccccgcgctggtttcgtctgtgcggcctcaagccttggcggcggtgcccatgaaagctcactttgtgagcaacttcgaggcatcatctcagagggcggggcagcagtcgcttcaagtgtctttgtcgctaccgtcaaatctttaggccccaggacacaacaactccaaattgaagactggctcgctttacttgaagacgagtatctgagtgaggagatgatggaactgacggcacgggctctcgagcggggaaatggcgaatggtccacagatgcagctttggaagtggcccacgaggcggaagcgctggtttcacaattgggtaatgcgggagaagtgtttaatttcggggacttcgggtgtgaggatgataatgcgaccccatttggcggccctggagccccagggccagcatttgctgggagaaagagagctttccacggagatgatccatttggtgaaggtccgccagacaagaagggagatctgacgctggatatgctc

SEQ ID NO: 33 - protein, 1196 aa, synthetic construct
  REGION 1..1196: note = ICP8 protein from HSV1
METKPKTATTIKVPPGPLGYVYARACPSEGIELLALLSARSGDSDVAVAPLVVGLTVESGFEANVAVVVGSRTTGLGGTAVSLKLTPSHYSSSVYVFHGGRHLDPSTQAPNLTRLCERARRHFGFSDYTPRPGDLKHETTGEALCERLGLDPDRALLYLVVTEGFKEAVCINNTFLHLGGSDKVTIGGAEVHRIPVYPLQLFMPDFSRVIAEPFNANHRSIGEKFTYPLPFFNRPLNRLLFEAVVGPAAVALRCRNVDAVARAAAHLAFDENHEGAALPADITFTAFEASQGKTPRGGRDGGGKGAAGGFEQRLASVMAGDAALALESIVSMAVFDEPPTDISAWPLFEGQDTAAARANAVGAYLARAAGLVGAMVFSTNSALHLTEVDDAGPADPKDHSKPSFYRFFLVPGTHVAANPQVDREGHVVPGFEGRPTAPLVGGTQEFAGEHLAMLCGFSPALLAKMLFYLERCDGAVIVGRQEMDVFRYVADSNQTDVPCNLCTFDTRHACVHTTLMRLRARHPKFASAARGAIGVFGTMNSMYSDCDVLGNYAAFSALKRADGSETARTIMQETYRAATERVMAELETLQYVDQAVPTAMGRLETIITNREALHTVVNNVRQVVDREVEQLMRNLVEGRNFKFRDGLGEANHAMSLTLDPYACGPCPLLQLLGRRSNLAVYQDLALSQCHGVFAGQSVEGRNFRNQFQPVLRRRVMDMFNNGFLSAKTLTVALSEGAAICAPSLTAGQTAPAESSFEGDVARVTLGFPKELRVKSRVLFAGASANASEAAKARVASLQSAYQKPDKRVDILLGPLGFLLKQFHAAIFPNGKPPGSNQPNPQWFWTALQRNQLPARLLSREDIETIAFIKKFSLDYGAINFINLAPNNVSELAMYYMANQILRYCDHSTYFINTLTAIIAGSRRPPSVQAAAAWSAQGGAGLEAGARALMDAVDAHPGAWTSMFASCNLLRPVMAARPMVVLGLSISKYYGMAGNDRVFQAGNWASLMGGKNACPLLIFDRTRKFVLACPRAGFVCAASSLGGGAHESSLCEQLRGIISEGGAAVASSVFVATVKSLGPRTQQLQIEDWLALLEDEYLSEEMMELTARALERGNGEWSTDAALEVAHEAEALVSQLGNAGEVFNFGDFGCEDDNATPFGGPGAPGPAFAGRKRAFHGDDPFGEGPPDKKGDLTLDML

SEQ ID NO: 34 - DNA, 66 bp, synthetic construct
  source 1..66: mol_type = other DNA
  misc_feature 1..66: note = Nucleotide sequence of the polynucleotide encoding the self-cleaving peptide P2A
ggctccggagctactaacttcagcctgctgaagcaggctggagatgtggaggagaaccctggacct

SEQ ID NO: 35 - DNA, 15034 bp, synthetic construct
  source 1..15034: mol_type = other DNA
  misc_feature 1..15034: note = Plasmid Ad-HBoV-HSV5
atctctatggcagggcctgccgccccgacgttggctgcgagccctgggccttcacccgaacttggggggtggggtggggaaaaggaagaaacgcgggcgtattggccccaatggggcctcggtggggtatcgacagagtgccagccctgggaccgaaccccgcgtttatgaacaaacgacccaacaccgtgcgttttattctgtctttttattgccgtcatagcgcgggttccttccggtattgtctccttccgtgtttcagttagcctcccccacttttagagcatatccagcgtcagatctcccttcttgtctggcggaccttcaccaaatggatcatctccgtggaaagctctctttctcccagcaaatgctggccctggggctccagggccgccaaatggggtcgcattatcatcctcacacccgaagtccccgaaattaaacacttctcccgcattacccaattgtgaaaccagcgcttccgcctcgtgggccacttccaaagctgcatctgtggaccattcgccatttccccgctcgagagcccgtgccgtcagttccatcatctcctcactcagatactcgtcttcaagtaaagcgagccagtcttcaatttggagttgttgtgtcctggggcctaaagatttgacggtagcgacaaagacacttgaagcgactgctgccccgccctctgagatgatgcctcgaagttgctcacaaagtgagctttcatgggcaccgccgccaaggcttgaggccgcacagacgaaaccagcgcggggacaggcaagtacaaattttcgagtccggtcgaaaatcagcagcgggcaggcgttcttaccgcccatcagagaagcccagttgccggcttgaaacacacgatcatttcctgccatgccgtaatacttagatatgctcaagcctaaaacgaccattggtcttgccgccattacaggtctaagcaagttacaactcgcaaacatacttgtccaagcgccaggatgtgcatccactgcgtccatcagagcacgtgcgcctgcttccagcccagcaccaccttgcgctgaccaagcggctgctgcttgaactgaagggggccggcggctacccgctatgatagcggtaagcgtatttatgaagtatgttgagtgatcacagtacctcaggatttggttagccatatagtacatagccaattcagaaacattgttaggagccaagttaatgaagtttatggcgccgtagtcgagggaaaatttcttgatgaacgcgatagtctcaatatcctcgcgggacaggagccgtgctggcagctgattgcgttgcaaggcggtccaaaaccattgcggattaggttgattggagccaggtggtttcccattggggaaaattgctgcgtggaattgtttgagcaagaatcccaacggcccaagcaggatatcgactctcttatcaggcttttggtaggcgctctgcagacttgcaactctagccttggcggcttcagaggcatttgcagaagcgccagcaaacaggaccctggacttgactctaagttccttgggaaagccgagggtgactcgcgcgacatcaccttcgaagcttgattcagctggggcggtttgcccagcagtaaggctgggtgcgcatatggctgcgccctctgacagagccactgtgagagttttggcggagaggaacccattattgaacatatccatcactcgccttctcagtactggttgaaattggttcctgaaattgcgaccttcgacggattgtccggcgaaaactccatgacattgggaaagggcgagatcctggtacaccgctaaattgctcctgcgccccagaagttgcagcagtggacatggcccacacgcatatggatcgagcgtcagagacatagcatggttagcttcgcccagaccgtccctaaatttgaaattcctgccttcaacgaggtttctcataagttgttcgacttcgcgatccaccacttgccggacgttgtttacgacggtgtgcaaggcttccctgttggtaataattgtctcaaggcgtcccatggcggtgggcactgcttgatcaacatattggagcgtttccaactcggccataacgcgttcagttgcagcgcgatacgtttcttgcataatcgttcttgctgtttctgatccgtcagctcttttaagagctgaaaatgcggcgtaattccccagtacatcgcaatcggaatacattgagttcatcgttccgaatacaccaatggcacccctggcagcactcgcaaatttaggatgtctagcgcgcaggcgcatgagagttgtgtggacgcaagcatggcgtgtgtcgaatgtacagagattgcaaggcacatcagtttgattgctgtctgccacatatcggaagacgtccatctcctgtctgccgactatcacggccccgtcacacctctcgagatagaacaacatcttagcaaggagtgccggagagaagccgcaaagcattgcaaggtgctctcccgcaaattcttgagtgccgcccaccagcggagctgtgggtcttccctcaaagccagggaccacatgtccttcccggtcaacttgaggattcgctgccacatgagtgcctggtaccaggaagaacctgtaaaagctgggtttggaatgatctttagggtcggctgggcccgcgtcatcgacctcggtcagatgaagggcgctattggtgctgaacaccatcgctccgaccagtccagctgcccgggccaaatatgcgccaacggcgttggcgcgcgctgcagcagtatcctgcccttcaaacagtggccaggcggaaatgtcggttgggggttcatcgaaaacggccatggatactatgctctccagtgccagtgccgcatctccggccatgacagaggctaatctctgctcaaagccgccggctgcgcctttaccaccaccgtcccgtccgccgcggggagttttgccttgactcgcctcaaaggcggtaaatgttatgtctgcgggcagtgccgcaccttcgtggttttcatcaaaggccaaatgggctgcagctcttgctaccgcatcgacattcctacacctcagtgctaccgcagctgggccgaccacagcttcaaacagcaaccggttaaggggcctgttaaagaaggggagagggtaggtaaacttttctcctatggacctgtgattggcattgaatggctcggcgatcaccctggaaaagtccggcatgaagagctgaagcgggtagacggggattcggtgaacctcagcgccaccgattgtcactttgtcgctaccgcccaaatgcagaaaagtattattgatgcagacggcctctttaaaaccttcggttaccacgaggtacagcaacgcacgatcgggatccagcccaagtctttcacaaagtgcctctcccgtagtttcatgcttcagatcaccaggtctgggcgtatagtcactaaagccaaaatgtcgccgggccctttcgcagagcctggtgagattaggtgcctgcgttgagggatcaaggtggcgcccaccgtggaagacgtatacagaagatgaatagtgactgggtgtcagtttaagggaaactgcagttccgccaagccccgtggttcttgagcccacgaccacagcaacattagcctcgaacccactttctacggtaagccccaccacgagtggggccactgcgacatcagagtccccgcttcgggcactcaacaaggccaacaattctatcccctcgctaggacaggcccgagcatatacgtaccccaggggtccagggggcaccttgatagtcgttgctgttttgggcttggtttccatggtggcctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccactgacgggcaccggagccgcctccatagagcccaccgcatccccagcatgcctgctattgtcctcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaccgttactgtctcttctcgccgatgtactcgccctcgtcctcgtcgctgggtctcaggtcctcgaagcagtgcagcacgatggggggctggttgggggtgccgctgctgatgaagcccagtctctcgccccactgggtggggtcgggctggggctcctgctcctggctgctgttgtcggccagggtctcctcctcgcctctctttctgctctctctcttcttctgcagcaggctggcctgtctctcgtactctctcagctcgtcctcgtccctgggctgggcgtgccaggtgttgaagaaggtgatggtgctcacgctgctggcggggctgttgggggggctggcgggggcggtcagtcttctcttcttgggggggcagtagtccacctggctggtctcgggctcggtgtcctcgccgtcgctgtcgcccaggtcgctgggggtgatgtcgccggtgctggtctcgctggctctgtcggtgtacatgctgttgtcggcgctgtggggcaggtagccgccgcagtcggtgcagtagccgttctcgtgcagggtgaagtcctcgggcaggtgctcccaggggcctctgccgccgtaggccatggtctgggggccggggatcacctcgttgtccagggtggcgtggtagttctttctgtagtgctcgggcaggggcaggccgttgatcagggtgtggctgtaggtcttctcggccaggatgaaccagttgtcggggctggtgcacacgtcgggcttgctgtagctgctgatgcatctgttcttgggcagcaggtagttcttgaagaatctcagggggtccacggtctgggcgtgcaggtcgcctcttctgtctctgtactgcaggatctgcatgttgccgccggtcacgccgccccaggcctctctctcggccttcttcagggtgtggaagatgtcggcctcctcgggctcgaagggtctggtggccagcaggctcttgcatctctggatgatctcggccaggatcaggccgtagaactgggcgcagctgctcttggcgtttctcttgctcaggccctcgccgcccacgatgatgtggcagtgcaggttcttctcgcccagctcgctctgcacgatgatgtcgcatctggggaacatgtttctctgcttgtggctcatcagggtggtggtggccatgtacacggccttgcacagcacggcgccgaagtgtctgtcctccaggctgatcagcaggtcgtccctgatgatgtcctcgtcggggtggggggcgcagtttctcagctggatcatgctctgctcggtgccgtgggccagcagggcgtgcagcagccactccttctccttccactgggggtaggggaacttgaacacgtaggtgaaggcgggctggctgaaggctctgatcacgggggggttgaaggccatggtggctctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgttttggtcgggacgctctggccggtcaggcgcgcgcaatcgttgacgctctagcgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagcccctaaaatttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagatgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcaaacgttattttggattgaagccaatatgataatgagggggtggagtttgtgacgtggcgcggggcgtgggaacggggcgggtgacgtaggttttagggcggagtaacttgtatgtgttgggaattgtagttttcttaaaatgggaagtgacgtaacgtgggaaaacggaagtgacgatttgaggaagttgtgggttttttggctttcgtttctgggcgtaggttcgcgtgcggttttctgggtgttttttgtggactttaaccgttacgtcattttttagtcctatatatactcgctctgcacttggcccttttttacactgtgactgattgagctggtgccgtgtcgagtggtgtttttttaataggttttcttttttactggtaaggctgactgttatgactacgtccggcgttccatttggcatgacactacgaccaacacgatctcggttgtctcggcgcactccgtacagtagggatcgtctacctccttttgagacagaaacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctaacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgtatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgccttatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtaggcgtggacttctccttcgccgcccgttaagcaaccgcaagttggacagcagcctgtggctcagcagctggacagcgacatgaacttaagtgagctgcccggggagtttattaatatcactgatgagcgtttggctcgacaggaaaccgtgtggaatataacacctaagaatatgtctgttacccatgatatgatgctttttaaggccagccggggagaaaggactgtgtactctgtgtgttgggagggaggtggcaggttgaatactagggttctgtgagtttgattaaggtacggtgatctgtataagctatgtggtggtggggctatactactgaatgaaaaatgacttgaaattttctgcaattgaaaaataaattttcgcggaacccctatttgtttatttttctaaatacattcaaatatgtatccgctcatgagacaataaccctgataaatgcttcaataatattgaaaaaggaagagtgaaggagaatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaataaccccttggggcctctaaacgggtcttgaggggttttttgatccaggcatcaaataaaacgaaaggctcagtcgaaagactgggcctttcgttttatctgttgtttgtcggtgaacgctctctactagagtcacactggctcaccttcgggtgggcctttctgcgtttatatagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccagagaatataaaaagccagattattaatccggcttttttattattt

SEQ ID NO: 36 - DNA, 11651 bp, synthetic construct
  source 1..11651: mol_type = other DNA
  misc_feature 1..11651: note = Plasmid Ad-HBoV5
aagtttactgtctcttctcgccgatgtactcgccctcgtcctcgtcgctgggtctcaggtcctcgaagcagtgcagcacgatggggggctggttgggggtgccgctgctgatgaagcccagtctctcgccccactgggtggggtcgggctggggctcctgctcctggctgctgttgtcggccagggtctcctcctcgcctctctttctgctctctctcttcttctgcagcaggctggcctgtctctcgtactctctcagctcgtcctcgtccctgggctgggcgtgccaggtgttgaagaaggtgatggtgctcacgctgctggcggggctgttgggggggctggcgggggcggtcagtcttctcttcttgggggggcagtagtccacctggctggtctcgggctcggtgtcctcgccgtcgctgtcgcccaggtcgctgggggtgatgtcgccggtgctggtctcgctggctctgtcggtgtacatgctgttgtcggcgctgtggggcaggtagccgccgcagtcggtgcagtagccgttctcgtgcagggtgaagtcctcgggcaggtgctcccaggggcctctgccgccgtaggccatggtctgggggccggggatcacctcgttgtccagggtggcgtggtagttctttctgtagtgctcgggcaggggcaggccgttgatcagggtgtggctgtaggtcttctcggccaggatgaaccagttgtcggggctggtgcacacgtcgggcttgctgtagctgctgatgcatctgttcttgggcagcaggtagttcttgaagaatctcagggggtccacggtctgggcgtgcaggtcgcctcttctgtctctgtactgcaggatctgcatgttgccgccggtcacgccgccccaggcctctctctcggccttcttcagggtgtggaagatgtcggcctcctcgggctcgaagggtctggtggccagcaggctcttgcatctctggatgatctcggccaggatcaggccgtagaactgggcgcagctgctcttggcgtttctcttgctcaggccctcgccgcccacgatgatgtggcagtgcaggttcttctcgcccagctcgctctgcacgatgatgtcgcatctggggaacatgtttctctgcttgtggctcatcagggtggtggtggccatgtacacggccttgcacagcacggcgccgaagtgtctgtcctccaggctgatcagcaggtcgtccctgatgatgtcctcgtcggggtggggggcgcagtttctcagctggatcatgctctgctcggtgccgtgggccagcagggcgtgcagcagccactccttctccttccactgggggtaggggaacttgaacacgtaggtgaaggcgggctggctgaaggctctgatcacgggggggttgaaggcaggtccagggttctcctccacatctccagcctgcttcagcaggctgaagttagtagctccggagccattggaggcatctgcttccatgctttcagcagcagaaagcatttcctcgtcagttatctcgtctgcttctgtctgtgaggaaacattagctaagtgtctacggtacacatcatcccagtaattacatctttcacaatcagagttatttctaaagtgccagaacatatttctgtatttttgatctagtgttttcttttgattaaacaatagttctctagtgttatcccatcctattttattatcaatttgtagctgttgaaactgttgtttcatttcattaaagattgaattagtaccatctctagcaatgcgagtagagtgccagtagaacccacaccaccctggagcttcaggattggaagctctgtgttgactgaatacagtgtatggatttgttctattgtcttttttccccgatgtactctccctcgtcttcatcacttggtctgaggtcttcgaagcagtgcaagacgataggtggctgattgggtgttcctgatgatatgagcccgagcctctctccccactgtgtcggatcgggctgcggctcctgctcctgtgatgagttgtctgccagtgtctcttcctctcccctctttctggactcccttttcttttgtaggagcgatgcttgtctttcatattccctgagctcatggtggctctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgttttggtcgggacgctctggccggtcaggcgcgcgcaatcgttgacgctctagcgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagcccctaaaatttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagatgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcaaacgttattttggattgaagccaatatgataatgagggggtggagtttgtgacgtggcgcggggcgtgggaacggggcgggtgacgtaggttttagggcggagtaacttgtatgtgttgggaattgtagttttcttaaaatgggaagtgacgtaacgtgggaaaacggaagtgacgatttgaggaagttgtgggttttttggctttcgtttctgggcgtaggttcgcgtgcggttttctgggtgttttttgtggactttaaccgttacgtcattttttagtcctatatatactcgctctgcacttggcccttttttacactgtgactgattgagctggtgccgtgtcgagtggtgtttttttaataggttttcttttttactggtaaggctgactgttatgactacgtccggcgttccatttggcatgacactacgaccaacacgatctcggttgtctcggcgcactccgtacagtagggatcgtctacctccttttgagacagaaacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctaacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgtatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgccttatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtaggcgtggacttctccttcgccgcccgttaagcaaccgcaagttggacagcagcctgtggctcagcagctggacagcgacatgaacttaagtgagctgcccggggagtttattaatatcactgatgagcgtttggctcgacaggaaaccgtgtggaatataacacctaagaatatgtctgttacccatgatatgatgctttttaaggccagccggggagaaaggactgtgtactctgtgtgttgggagggaggtggcaggttgaatactagggttctgtgagtttgattaaggtacggtgatctgtataagctatgtggtggtggggctatactactgaatgaaaaatgacttgaaattttctgcaattgaaaaataaattttcgcggaacccctatttgtttatttttctaaatacattcaaatatgtatccgctcatgagacaataaccctgataaatgcttcaataatattgaaaaaggaagagtgaaggagaatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaataaccccttggggcctctaaacgggtcttgaggggttttttgacgttccaggcatcaaataaaacgaaaggctcagtcgaaagactgggcctttcgttttatctgttgtttgtcggtgaacgctctctactagagtcacactggctcaccttcgggtgggcctttctgcgtttatatagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccagagaatataaaaagccagattattaatccggcttttttattatttctatccatagagcccaccgcatccccagcatgcctgctattgtcctcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcg